(19) United States
(12) Patent Application Publication
(10) Pub. No.: US 2017/0313779 A1
(43) Pub. Date: Nov. 2, 2017

(54) METHODS AND COMPOSITIONS PERTAINING TO HUMAN CEREBRAL CAVERNOUS MALFORMATIONS

(71) Applicant: The University of Chicago, Chicago, IL (US)

(72) Inventor: Issam A. Awad, Chicago, IL (US)

(21) Appl. No.: 15/523,521

(22) PCT Filed: Nov. 5, 2015
(86) PCT No.: PCT/US15/59135

§ 371 Date: May 1, 2017

Related U.S. Application Data
(60) Provisional application No. 62/075,491, filed on Nov. 5, 2014.

(51) Int. Cl. C07K 16/28; A61K 38/17; A61K 45/06; A61K 9/00; A61K 31/551; A61K 39/395; A61K 39/00

(52) U.S. Cl. CPC C07K 16/2878; A61K 31/551; A61K 38/1774; A61K 39/3955; A61K 45/06; A61K 9/0019; A61K 2039/507; C07K 2317/21; C07K 2317/73; C07K 2319/30; C07K 2319/32; A61K 2039/505

(57) ABSTRACT

The disclosure is based on the discovery that aberrant immune responses are contributing to the pathogenicity of CCMs in patients, and that blocking those responses treats the CCM. Described herein is a method for treating CCMs in a patient in need thereof comprising administering a therapeutically effective amount of a B-cell immunomodulation therapy to the patient.

### CROSS-REFERENCE TO RELATED APPLICATIONS

This application claims the benefit of priority of U.S. Provisional Patent Application No. 62/075,491 filed Nov. 5, 2014, which is hereby incorporated by reference in its entirety.

This invention was made with government support under Grant No. R21 NS052285 and NIH/NINDS R01 NS077957 awarded by the National Institutes of Health. The government has certain rights in the invention.

### BACKGROUND OF THE INVENTION

### 1. Field of the Invention

The present invention relates generally to the field of medicine. More particularly, it concerns methods and compositions for treating cerebral cavernous malformations.

### 2. Description of Related Art

The cerebral cavernous malformation (CCM), a common cerebrovascular anomaly, predisposes patients to a lifetime risk of seizures, hemorrhagic stroke, and other neurological sequelae (Otten et al., 1989; Robinson et al., 1991; Robinson et al., 1993). The clinical manifestations of CCMs are highly related to hemorrhage and/or CCM expansion (Maraire and Awad, 1995). The genesis and progression of individual CCM lesions are largely unpredictable (Gault et al., 2004; Denier et al., 2006; Gault et al., 2006). Lesions occur sporadically, in association with venous anomalies (Guclu et al., 2005), or may be inherited as autosomal dominant traits, with 3 known genetic loci (Revencu and Vikkula, 2006). Despite progress in the characterization of the genetic basis of the disease (Laberge-le Couteulx et al., 1999; Sahoo et al., 1999; Liguori et al., 2003; Denier et al., 2004; Bergametti et al., 2005), the molecular mechanisms involved in the pathophysiology of CCM await characterization. Moreover, there is no known treatment to alter the course of this disease. Therefore, there is a need in the art for CCM therapeutics.

### SUMMARY OF THE INVENTION

Described herein are methods and compositions that can be used to treat cerebral cavernous malformations (CCMs). Aspects of the disclosure relate to methods for treating cerebral cavernous malformations (CCMs) in a patient determined to be in need thereof comprising administering a therapeutically effective amount of a B-cell immunomodulation therapy to the patient.

B-cell immunomodulation therapies are known in the art and described herein. In some embodiments, the B-cell immunomodulation therapy is a B-cell depletion therapy. In some embodiments, the B-cell immunomodulation therapy inhibits B and T cell interactions. In some embodiments, the B-cell immunomodulation therapy comprises an antibody. In some embodiments, the antibody is a human antibody, humanized antibody, recombinant antibody, chimeric antibody, an antibody derivative, a veneered antibody, a diabody, a monoclonal antibody, or a polyclonal antibody. In some embodiments, the antibody is a human antibody.

In some embodiments, the B-cell immunomodulation therapy comprises an antibody or molecule (polypeptide, nucleic acid, or small-molecule inhibitor) that targets B-cell-specific surface molecules and eliminates the B-cells. Examples of these include anti-CD20 therapy (rituximab, ocrelizumab, veltuzumab and of atumumab), anti-CD19 therapy (MDX-1342), anti-CD22 therapy (epratuzumab), and Abatacept. In certain embodiments, the agent is capable of depleting at least or at most 10, 20, 30, 40, 50, 60, 70, 80, 90 or more percent (or any range derivable therein) of exposed B-cells by an in vitro assay.

In some embodiments, the B-cell immunomodulation therapy comprises an antibody or molecule (polypeptide, nucleic acid, or small-molecule inhibitor) that blocks B-cell activation and/or survival. Examples of these include anti-BLyS antibodies (anti-BR3, or belimumab) and atacicept. In some embodiments, the B-cell immunomodulation therapy comprises a B-lymphocyte stimulator (BLyS)-specific inhibitor. In some embodiments, the B-cell immunomodulation therapy comprises atacicept or belimumab, or both. In some embodiments, the B-cell immunomodulation therapy comprises belimumab.

In some embodiments, the B-cell immunomodulation therapy comprises an antibody or molecule (polypeptide, nucleic acid, or small-molecule inhibitor) that targets B-cell specific surface molecules. Examples include anti-CD20 therapies, anti-CD19 therapies, and anti-CD22 therapies described herein.

In some embodiments, the method or pharmaceutical compositions described herein are for treating Stage II CCMs. In some embodiment treatment targets the genesis of and number of Stage I lesions. Stage I CCM lesions are characterized by dilated capillaries having the width at least 25 red blood cells and not joined to any other lesion (isolated caverns). Stage II lesions are characterized by multicavernous structures composed of the confluence of two or more caverns. Stage II lesions develop from the maturation of Stage I lesions, and are responsible for clinical sequelae. It is well within the skill of a physician to diagnose a Stage I or Stage II CCM lesion (by MRI, for example).

The CCM may be diagnosed as familial (determined to have a genetic pre-disposition due to a family history of CCM) or sporadic (no family history of CCM). In some embodiments, the CCM is sporadic. In some embodiments, the CCM is familial. A patient in need of treatment may be someone showing symptoms of CCM, someone diagnosed with CCM, someone whose medical or family history indicates a significant risk of CCM, and/or someone previously treated for CCM.

The methods described herein may also further comprise one or more additional treatments for CCM such as surgical resection, radiosurgery, or the administration of additional therapeutics described herein. The additional treatment may be performed or administered before, after, or concurrent with the B-cell immunomodulation therapy. When the additional treatment is a therapeutic, it may be administered in the same composition as the B-cell immunomodulation therapy or in a different composition. In some embodiments, the method further comprises surgical resection of the CCM. In some embodiments, the patient has been previously treated for CCM with radiosurgery or surgical resection. In some embodiments, the method further comprises radiosurgery. In some embodiments, the method further comprises administration of an additional immunomodulating agent. Immunomodulating agents are known in the art and described herein. In some embodiments, the additional immunomodulating agent is administered concomitantly. In some embodiments, the immunomodulating agent is a co-stimulation inhibitor. In some embodiments, the immunomodulating agent is abatacept. In some embodiments, the method further comprises administration of a Rho-kinase (ROCK) inhibitor, which also alters immune response and inflammation. In some embodiments, the Rho-kinase inhibitor is fasudil. In some embodiments, the method further comprises administration of a HMG-CoA reductase inhibitor. Non-limiting examples of HMG-CoA reductase inhibitors include statins. HMG-CoA reductase inhibitors have pleiotropic effects, which include RhoA/ROCK inhibition and are useful in the methods described herein. In some embodiments, the HMG-CoA reductase inhibitor is a statin, also shown to alter immune response in the lesions.

In some embodiments, the B-cell immunomodulation therapy comprises one or more of an antibody or molecule that blocks B-cell activation and/or survival; an antibody or molecule that targets B-cell specific surface molecules; a co-stimulatory inhibitor; a ROCK inhibitor; an HMG-CoA reductase inhibitor; or combinations thereof.

In some embodiments, the B-cell immunomodulation therapy comprises an antibody or molecule that blocks B-cell activation and/or survival and one or more of: an antibody or molecule that targets B-cell specific surface molecules; a co-stimulatory inhibitor; a ROCK inhibitor; or an HMG-CoA reductase inhibitor.

In some embodiments, the B-cell immunomodulation therapy comprises, consists of, or consists essentially of belimumab and fasudil.

The administration of the therapeutics according to the methods described herein may be performed by any suitable administration known in the art. In some embodiments, the B-cell immunomodulation therapy is administered parenterally. In some embodiments, the B-cell immunomodulation therapy is administered intravenously.

In some embodiments, the method further comprises monitoring the patient for B-cell immunomodulation. The patient may be monitored for B-cell immunomodulation by methods known in the art. For example, the monitoring of the patient may comprise magnetic resonance imaging (MRI) of the cranium. In some embodiments, serial MRI imaging may be performed to evaluate the CCM lesions. Evaluation of the CCM lesions may include evaluating the number, size, hemorrhage thereof, or iron accumulation therein. In some embodiments, monitoring the patient comprises evaluating the blood of the patient to determine whether the B-cells have been modulated. For example, blood may be drawn from the patient and screened for the presence of B cells. Methods for determining the presence of B cells are known in the art and described herein throughout the disclosure and especially in Example 2. The blood of the patient may also be evaluated for biomarker activity of CCM disease. An example of a biomarker for CCM disease includes ROCK activity.

Further aspects of the disclosure relate to a pharmaceutical composition comprising a B-cell depleting agent and a ROCK inhibitor. The B-cell depleting agent may be one as described herein. In some embodiments, the ROCK inhibitor is fasudil.

In some embodiments, the pharmaceutical composition further comprises a HMG-CoA reductase inhibitor. In some embodiments, the HMG-CoA reductase inhibitor is a statin.

The term “treating” as used herein is intended to encompass curing as well as ameliorating at least one symptom of the condition or disease. For example, a response to treatment may include a reduction in bleeding rate in lesions, iron content in lesions, seizures, prevention of disability, increase in survival time, elongation in time to lesion genesis or progression, reduction in lesion mass, reduction in lesion burden and/or a prolongation in time to lesion recurrence, lesion response, complete response, partial response, stable disease, progressive disease, progression free survival, or overall survival.

As used herein the specification, “a” or “an” may mean one or more. As used herein in the claim(s), when used in conjunction with the word “comprising”, the words “a” or “an” may mean one or more than one.

As used herein, the term “comprising” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would exclude additional active ingredients not listed in the claim, but would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention. When the term, “comprising” or “comprise” or “comprises” is used in methods or compositions herein, embodiments also include compositions and methods that “consist of” or “consist essentially of.”

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1C—Immune complex formation in CCM lesions. Antibody containing immune complex deposition was evident by co-localization (merge/C) of IgG (A) and MAC (B). Original magnification is 200×. All scale bars are 50 μm.

FIGS. 2A-2F—Antibody producing cells aggregating with T-cells in CCM lesions. CD20₊ B-cells (red) and CD3₊ T-cells (brown) were aggregated around a cavern of a CCM lesion (T:B aggregate) (A). CD138₊ plasma-cells (brown) were well-circumscribed around a cavern of a CCM lesion (B). CD4₊ helper T-cells were one of T-cell subtypes (C). CD8₊ cytotoxic T-cells were another T-cell subtype (D). CD4₊ helper T-cells (brown) and CD20₊ B-cells (red) (E) or cytotoxic CD8₊ T-cells (brown) and CD20₊ B-cells (red) (F) were co-localized in CCM lesions respectively. Original magnification is 400×. All scale bars are 50 μm.

FIG. 3—Alignment of predicted germ-line sequences with corresponding IgG H-chain variable regions expressed in CCM lesion. Nucleotide sequences matching to VH3_49*04 (H-chain) expressed transcripts from patient 9 were aligned with the germ-line predicted using IMGT (SEQ ID NO: 2). Identical nucleotides are indicated by dashes (SEQ ID NO: 3-5). Translated amino acid codons are also given (SEQ ID NO: 1). VH3_49*04=VH3-49*04 D1-1*01 J4*02.

FIGS. 4A-4B—In situ clonal expansion. Genealogical relationships of expressed IgG H- and L-chain variable region sequences shown in FIGS. 3 and 5 are illustrated as a clonal tree with the predicted germ-line at the top. A: IgG H-chain. B: IgG L-chain. The gradual accumulation and pattern of mutation are consistent with in situ somatic hypermutation.

FIG. 5—Alignment of germ-line sequences with IgG L-chain variable regions expressed in CCM. Nucleotide sequences of VL1_44*01 (L-chain) expressed transcripts from patient 9 were aligned with the germ-line obtained from the IMGT database. VL1_44*01=VL1-44*01 J2*01/J3*01 (SEQ ID NO: 6-10).

FIG. 6—Alignment of germ-line sequences with IgG H-chain variable regions expressed in CCM. Nucleotide sequences of VH5_51*01 (H-chain) amplified transcripts from patient 10 were aligned with the germ-line obtained from the IMGT database. VH5_51*01=VH5-51*01 D3-10*01 J4*01/J4*02/J4*03 (SEQ ID NO: 11-21).

FIGS. 7A-7B—In situ clonal expansion. Genealogical relationships of sequences shown in FIGS. 6 and 8 are illustrated in a clonal tree, with a predicted germ-line clone at the top. A: IgG H-chain in the CCM lesion from patient 10. B: IgG L-chain in the CCM lesion from patient 10. The gradual accumulation and pattern of mutation is consistent with in situ somatic hypermutation.

FIG. 8—Alignment of germ-line sequences with IgG L-chain variable regions expressed in CCM. Nucleotide sequences of VK1_9*01 (L-chain) amplified transcripts from patient 10 were aligned with the germ-line obtained from the IMGT database. VK1_9*01=VK1-9*01 J4*01 (SEQ ID NO: 22-33).

FIG. 9—Alignment of predicted germ-line sequences with corresponding IgG H-chain variable regions expressed in CCM lesion. Nucleotide sequences of VH1_2*02 (H-chain) amplified transcripts from patient 10 were aligned with the germ-line predicted using IMGT. Identical nucleotides are indicated by dashes. Translated amino acid codons are also given. VH1_2*02=VH1-2*02 D2-21*02 J6*02 (SEQ ID NO: 34-38).

FIG. 10—Alignment of predicted germ-line sequences with corresponding IgG H-chain variable regions expressed in CCM lesion. Nucleotide sequences of VH1_69*06 (H-chain) amplified transcripts from patient 10 were aligned with germ-line obtained from the IMGT database. VH1_69*06=VH1-69*06 D4-11*01 J4*02/J4*03 (SEQ ID NO: 39-43).

FIG. 11—In situ clonal expansion. Genealogical relationships of expressed IgG H-chain variable region sequences shown in Supplementary FIGS. 1 and 2 were illustrated in a clonal tree with a predicted germ-line clone at the top. A and B: IgG H-chains in the CCM lesion of patient 10. The gradual accumulation and pattern of mutation are consistent with in situ hypermutation.

FIG. 12—Alignment of predicted germ-line sequences with IgG L-chain variable regions expressed in CCM lesion. Nucleotide sequences of VK1_5*01 (L-chain) transcripts amplified from the lesion of patient 10 were aligned with the germ-line obtained from the IMGT database. VK1_5*01=VK1-5*01 J2*01/J2*02 (SEQ ID NO: 44-48).

FIG. 13—Alignment of predicted germ-line sequences with corresponding IgG L-chain variable regions expressed in CCM. Nucleotide sequences of VK1_33*01 (L-chain) transcripts amplified from the lesion of patient 10 were aligned with the germ-line obtained from the IMGT database. VK1_33*01=VK1-33*01NK1D-33*01 J1*01 (SEQ ID NO: 49-55).

FIG. 14—In situ clonal expansion. Genealogical relationships of expressed IgG L-chain variable region sequences shown in Supplementary FIGS. 4 and 5 were illustrated in a clonal tree with a predicted germ-line clone at the top. A and B: IgG L-chains in the patient 10 CCM lesion. The gradual accumulation and pattern of mutation are consistent with in situ hypermutation.

FIG. 15—B cell depletion from blood and spleen was confirmed in 5 out of 5 Ccm3^{+/-}Trp53^{-/-} mice. FIG. 9A are FACS plots showing B-cell depletion, as evidenced by the decrease in the number of CD45R⁺CD3⁻ and CD22⁺CD19⁺ cells. FIG. 9B graphically shows the data demonstrated in FIG. 9A from five mice.

FIG. 16—Depleted B cells were confirmed in spleen. Shown are histology sections of the spleen of control or B-cell depleted mice. The sections were immunohistochemically analyzed for B (top panel) and T cells (bottom panel).

FIG. 17—Representative lesions within the brain of a murine model for cerebral cavernous malformation disease in untreated controls and after B cell depletion.

FIG. 18—B cell depletion in Ccm3+/−Trp53−/− mice resulted in a significant lower burden of mature (stage II) CCM lesions, but had a lesser effect on capillary ecstasia (stage 1) pre-lesions.

FIG. 19—B cell depleted Ccm3+/−Trp53−/− mice exhibited significant smaller CCM lesions area (effect on mature CCM lesions).

FIG. 20—No B cells were detected in the lesions of Ccm3+/−Trp53−/− mice treated with anti-BR3 antibody.

FIG. 21—Tumors in B cells depleted mice. Extra-cranial tumors were found in 3 out of 5 B-cell depleted Ccm3+/−Trp53−/− mice. No tumors were detected any tumors in 10 out of 10 B-cell deleted Ccm3+/− mice, indicating Trp53−/− background increase the risk of tumors with B cell depletion.

FIG. 22—Lesional area per animal in Ccm3+/− mice with B cell depletion. The lesional area per animal was significantly lower in the more mature clinically significant stage 2 lesions in B cell depleted mice. This proves the benefit of B cell depletion in a heterozygous Ccm model without concomitant Trp53−/− background, more closely recapitulating the human disease.

FIG. 23—Number of lesions per animal in Ccm3+/− mice with B cell depletion. The prevalence of CCM lesions was significantly lower in B-cell depleted mice, and a significant decrease in the more mature clinically significant stage 2 lesions is observed in B-cell depleted mice. This proves the benefit of B cell depletion in a heterozygous Ccm model without concomitant Trp53−/− background, more closely recapitulating the human disease.

### DESCRIPTION OF ILLUSTRATIVE EMBODIMENTS

The disclosure is based on the discovery that aberrant immune responses are contributing to the pathogenicity of CCMs in patients, and that blocking those responses effects a treatment of the CCM. Described herein is a method for treating CCMs in a patient in need thereof comprising administering a therapeutically effective amount of a B-cell immunomodulation therapy to the patient.

### I. B-CELL IMMUNOMODULATION THERAPY

One strategy for modulating B cells is through the use of antibodies or inhibitors that target B-cell-specific surface molecules or B-cell survival factors or through the use of antibodies or inhibitors that disrupt B and T cell interaction. Examples of such antibodies and/or B-cells specific surface molecules are described below.

B. BLyS

B-lymphocyte stimulator (BLyS) (also known as B-cell activating factor (BAFF), RP11-153124.1, CD257, DTL, TALL-1, TALL1, THANK, TNFSF20, ZTNF4, and TNSF13B). B lymphocytes (B cells) are one of the immune cells responsible for the damage in autoimmune disease. B cells develop in the bone marrow and continue to mature peripherally in secondary lymphoid organs. When autoimmune B cells attack the body's own tissues, they are normally destroyed by apoptosis. In order to survive, B cells need survival factors. It is believed that autoimmunity in some diseases may be caused when autoimmune B cells proliferate, and survival factors protect them from cell suicide.

Accordingly, antibodies or inhibitors that target BLyS may be used for B-cell depletion/immunomodulation therapy. In one embodiment, the B-cell immunomodulation therapy is an anti-BLyS antibody. In one embodiment, the B-cell immunomodulation therapy is belimumab. Belimumab (trade name Benlysta, previously known as LymphoStat-B) is a human monoclonal antibody that inhibits BLyS. Belimumab binds to BLyS, preventing

BLyS from binding to B cells. In another embodiment, the B-cell immunomodulation therapy is tabalumab. In yet another embodiment, the B-cell immunomodulation therapy is blisibimod. Without being limited to any scientific theory, it is believed that, without the survival factor BLyS, B cells become apoptotic, and no longer contribute to CCM pathogenicity.

C. CD20

B-lymphocyte antigen CD20 (CD20) is primarily found on the surface of B-cells beginning at the pro-B phase and progressively increasing in concentration until maturity. In humans, CD20 is encoded by the MS4A1 gene. Therefore, antibodies and therapeutics that target and effectively eliminate CD20-expressing cells are useful in B-cell immunomodulation therapy. One such antibody is rituximab. In some embodiments, the B-cell immunomodulation therapy is an anti-CD20 antibody. In some embodiments, the B-cell immunomodulation therapy is rituximab. Rituximab (trade names Rituxan, MabThera and Zytux) is a chimeric monoclonal antibody against the protein CD20. Rituximab destroys B cells and is therefore used to treat diseases which are characterized by excessive numbers of B cells, overactive B cells, or dysfunctional B cells. Other anti-CD20 antibodies useful as B-cell immunomodulation therapies include ocrelizumab and ofatumumab.

D. CD19

CD19 is a B-cell-specific membrane protein that is broadly expressed during B-cell development, from the pro-B cell to the early plasma cell stage. Therefore, anti-CD19 antibodies may be used as the B-cell immunomodulation therapy. In one embodiment, the anti-CD19 antibody is the humanized mouse HB12 anti-hCD19 mAb. In some embodiments, the anti-CD19 antibody is an Fc-engineered, affinity-matured humanized anti-hCD19 mAb, which may have increased FcγR binding affinity and has been shown to improve antibody-dependent cellular cytotoxicity. In some embodiments, the anti-CD19 antibody is MDX-1342 (Medarex, Princeton, N.J.), which is a fully humanized antibody that not only binds selectively to CD19 expressed on B cells (without targeting stem cells or fully differentiated plasma cells, which lack CD19 expression), but that also induces the immunomodulation and elimination of CD19-positive B cells.

E. CD22

CD22 is a member of the Ig superfamily and binds to sialic acid-bearing molecules on other hematopoietic and non-hematopoietic cells. In some embodiments, the B-cell immunomodulation therapy is an anti-CD22 antibody. In some embodiments, the anti-22 antibody is epratuzumab. Epratuzumab is a monoclonal antibody against CD22 that inhibits B-cell proliferation.

F. Atacicept

In some embodiments, the B-cell immunomodulation therapy is atacicept. Atacicept is a fully humanized, recombinant receptor-Ig fusion protein (TACI-Ig or atacicept). Atacicept binds to the B-cell growth factors BLyS and APRIL. Atacicept inhibits B-cell maturation, differentiation, and survival, as well as immunoglobulin production, by depriving B cells of needed growth and development signals.

G. Abatacept

In some embodiments, the B-cell immunomodulation therapy is Abatacept. Abatacept, also known as CTLA4-Ig is a recombinant fusion protein that blocks the co-stimulatory signal mediated by the CD28-CD80/86 pathway and disrupts T and B cell interaction. Simple signaling without co-stimulation allows the cell to recognize the primary signal as “self” and not ramp-up responses for future responses as well. Accordingly, in some embodiments, the method further comprises administration of a co-stimulatory inhibitor.

### II. PHARMACEUTICAL COMPOSITIONS

The term “administration” shall include without limitation, administration by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration.

Compositions may be conventionally administered parenterally, by injection, for example, intravenously, subcutaneously, or intramuscularly. In some embodiments, of the methods and compositions described herein, the administration is done intravenously.

Additional formulations which are suitable for other modes of administration include oral formulations. Oral formulations include such normally employed excipients such as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%.

Typically, compositions are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner. However, suitable dosage ranges are of the order of ten to several hundred nanograms or micrograms antigen-MHC-nanoparticle complex per administration. Suitable regimes for initial administration and boosters are also variable, but are typified by an initial administration followed by subsequent administrations.

In some embodiments, the dosage of the B-cell depleting therapy is 1-100 mg/kg or 1 to 10, 20, 30, 40, 50, 60, 70, 80, 90, or 200 mg/kg or any range derivable therein. In some embodiments, the dosage is 10 mg/kg. In some embodiments, the dosage is 1, 5, 10, 20, 30, 40, 50, 60, 70, 80, 90, or 100 mg/kg.

In many instances, it will be desirable to have multiple administrations of the B-cell depleting therapy, about, at most about or at least about 3, 4, 5, 6, 7, 8, 9, 10 or more. The administrations may range from 2 day to twelve week intervals, or from one to two, three, four, five, six, eight, twelve, or sixteen week intervals. In some embodiments, the B-cell immunomodulation therapy is administered at two-week intervals for the first 1, 2, 3, 4, 5, 6, 7, or 8 doses. In some embodiments, the therapy is administered at four-week intervals for doses 1, 2, 3, 4, 5, 6, 7, 8, 9, 10, or more.

In some embodiments, the B-cell depleting therapy is administered at 2-week intervals for the first three doses and at 4-week intervals for any subsequent dose.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier may be a solvent or dispersion medium containing, for example, water, ethanol, polyol (for example, glycerol, propylene glycol, and liquid poly(ethylene glycol), and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by sterilization. Sterilization of the solution will be done in such a way as to not diminish the therapeutic properties of the active ingredients. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterilized solution thereof.

Embodiments of the disclosure include unit doses of the pharmaceutical compositions. An effective amount of therapeutic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

### III. COMBINATION THERAPY

The compositions and related methods described herein may also be used in combination with the administration of additional therapies such as additional immunomodulating agents or additional medical procedures, in the case of the methods described herein. For example, the method may include surgical resection of the CCM prior to or after B-cell immunomodulation therapy. Surgical resection comprises surgery that removes abnormal tissue, or in this case, tissue characterized as a CCM. Radiosurgery may also be performed prior to or after B-cell immunomodulation therapy. Radiosurgery is a non-invasive, radiation based treatment. In some cases, the radiosurgery may be stereotactic radiosurgery. Stereotactic radiosurgery (SRS) treats brain disorders with a precise delivery of a single, high dose of radiation in a one-day session. Focused radiation beams are delivered to a specific area of the brain to treat abnormalities, tumors or functional disorders.

The compositions and methods described herein may also include additional immunomodulators such as immunosuppressants known in the art. Non-limiting examples include cortisone, cyclosporine, azathioprine, ciclosporin, tacrolimus, and sirolimus. The compositions and methods may also include the administration of co-stimulatory inhibitors. A non-limiting example of a co-stimulatory inhibitor is abatacept. Abatacept prevents antigen-presenting cells (APCs) from delivering the co-stimulatory signal. This prevents the T cells from being fully activated, and even downregulates them. Simple signaling without co-stimulation allows the cell to recognize the primary signal as “self” and not ramp-up responses for future responses as well. Accordingly, in some embodiments, the method further comprises administration of abatacept or a co-stimulatory inhibitor.

The compositions and methods described herein may also include a ROCK inhibitor. Rho-associated protein kinase (ROCK) is a kinase belonging to the AGC (PKA/PKG/PKC) family of serine-threonine kinases. It is involved mainly in regulating the shape and movement of cells by acting on the cytoskeleton. In some embodiments, the ROCK inhibitor is fasudil.

The compositions and methods described herein may also include a HMG-CoA reductase inhibitor. Non-limiting examples of HMG-CoA reductase inhibitors include statins. HMG-CoA reductase inhibitors such as statins have pleiotropic effects that include RhoA/ROCK inhibition, and are therefore useful in the compositions and methods described herein. Non-limiting examples of statins include atorvastatin, fluvastatin, lovastatin, pitavastatin, pravastatin, rosuvastatin, and simvastatin.

When combination therapy is employed, various combinations may be employed, for example B-cell depleting agent/therapeutic administration is “A” and the additional agent is “B”, administration may be: A/B/A, B/A/B, B/B/A, A/A/B, AB/B, B/A/A, A/B/B/B, B/A/B/B, B/B/B/A, B/B/A/B, A/A/B/B, A/B/A/B, A/B/B/A, B/B/A/A, B/A/B/A, B/A/A/B, A/A/A/B, B/A/A/A, A/B/A/A, or A/A/B/A.

Administration of the therapeutics described herein to a patient/subject will follow general protocols for the administration of such compounds, taking into account the toxicity, if any. It is expected that the treatment cycles would be repeated as necessary. It also is contemplated that various standard therapies, such as hydration, may be applied in combination with the described therapy.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Immune Complex Formation and In Situ B-Cell Clonal Expansion in Human Cerebral Cavernous Malformations

The cerebral cavernous malformation (CCM), a common cerebrovascular anomaly, predisposes patients to a lifetime risk of seizures, hemorrhagic stroke, and other neurological sequelae (Often et al., 1989, Robinson et al., 1991, Robinson et al., 1993). The clinical manifestations of CCMs are highly related to hemorrhage and/or CCM expansion

(Maraire and Awad, 1995). The genesis and progression of individual CCM lesions are largely unpredictable (Denier et al., 2006, Gault et al., 2006, Gault et al., 2004). Lesions occur sporadically, in association with venous anomalies (Guclu et al., 2005), or may be inherited as autosomal dominant traits, with 3 known genetic loci (Revencu and Vikkula, 2006). Despite progress in the characterization of the genetic basis of the disease (Bergametti et al., 2005, Denier et al., 2004, Laberge-le Couteulx et al., 1999, Liguori et al., 2003, Sahoo et al., 1999), the molecular mechanisms involved in the pathophysiology of CCM await characterization. Moreover, there is no known treatment to alter the course of this disease.

The human CCM lesion consists of clusters of cavernous anomalous vessels, lined by endothelium, and filled with blood at various stages of thrombosis and organization. A defective blood-brain barrier has been observed in CCMs (Tu et al., 2005, Wong et al., 2000), and lesions characteristically include chronic deposition of blood breakdown products, including iron in heme and non-heme forms (Bradley, 1993, Rigamonti et al., 1987). Invariably, the lesions exhibit hallmarks of blood leakage and gliotic reaction in adjacent brain. The presence of a unique immune response in these lesions is compelling, given the characteristic vascular phenotype with the sequestration of thrombi at various stages of organization, leaky blood-brain barrier, and the chronic deposition of iron and blood degradation products (Rigamonti et al., 1987, Steiger et al., 1987). It is hypothesized that these factors create a neo-antigenic milieu in CCM lesions that may drive the activation of non-tolerized lymphocytes and, ultimately, mediate the pathological inflammatory response in situ.

Applicants' studies revealed robust inflammatory cell infiltration in CCM lesions, with prominent antibody producing cells (CD20+ B-cells and CD138+ plasma-cells), CD3+ T-cells, CD68+ antigen presenting macrophages, and the MHC class II antigen presentation molecule HLA-DR. These observations are consistent, regardless of history of recent clinically overt hemorrhage or recent growth. B-cells and plasma-cells were found to be present in all lesion types, including familial and sporadic cases (Shi et al., 2009). They were also present in genetically engineered mice that develop lesions that recapitulate those found in human CCMs (McDonald et al., 2011, McDonald et al., 2012). However, the pathological significance of the infiltrate was unknown.

Applicants' isoelectric focusing results showed that CCM lesions had oligoclonal patterns of IgG unrelated to peripheral blood contamination, indicating selective synthesis of IgG within the lesions (Shi et al., 2007). These findings raise the possibility that an in situ clonal expansion of B cells that could differentiate into pathogenic antibody producing plasma-cells. Applicants' findings from spectratyping, cloning, and sequencing of CCM lesions further demonstrated amplification of particular IgG heavy (H) chain CDR3 regions. These regions were significantly different from those demonstrated for peripheral blood lymphocytes (Shi et al., 2009). Despite the discoveries listed above, in situ antigen-driven B-cell clonal expansion in CCM lesions remain undetermined. Knowledge of the clonal relatedness of variable regions expressed in situ would provide more definitive evidence of in situ B-cell clonal expansion and antigen-driven affinity maturation. Moreover, comparison of genetic data from antibody variable regions expressed in situ by different patients would also help discern whether antibody responses (and therefore targeted antigens) are common between patients. Ultimately, provision of these data, together with direct evidence of antibody mediated pathogenesis, via either complement or Fc receptor activation, would support Applicants' hypothesis that within CCM lesions, a synergistic relationship between B- and T-cells mediates antigen-driven B-cell clonal expansion, antibody production, and local antibody dependent activation of the complement pathway. Should this be the case, inhibition of pathways that mediate autoimmunity could be a potential therapeutic strategy in CCMs. More specifically, detailed characterization of the molecular and cellular interplay leading to this autoimmune response would elucidate exactly which targets are most relevant for therapeutic intervention.

In the study described in this example, the following material and methods were used.

Patients. This study included 10 patients (patients 1-10) undergoing surgical excision of CCMs for clinical indications unrelated to the research study. The diagnosis of CCM was established by evidence of typical histopathological criteria in every case. The study was approved by the University of Chicago institutional review board. All participants gave informed consent for the processing of a portion of their surgical specimens for research purposes.

Co-localization of IgG and the complement membrane attack complex to demonstrate antibody dependent immune complex formation in human CCM lesions. CCM lesions were examined for deposition of IgG and complement protein by tissue immunofluorescence. Five micron cryo-sections from frozen human CCM specimens from patients 1-6 were double stained with anti-IgG antibody conjugated to FITC (Invitrogen,1:100) and mouse anti-C5b-9/membrane attack complex MAC (Dako, M0777, aE11, 1:50), followed by anti-mouse IgG secondary antibody conjugated to AlexaFluor 546 (Invitrogen,1:1000). Isotype controls were used as negative controls, and run simultaneously with all the specimens. Co-localization of IgG (green) and the complement (red) was visualized by immunofluorescent microscopy (Olympus BH-2 microscope with BH2-RFC reflected light fluorescence attachment).

Immunohistochemistry. To detect CD20⁻ B- and CD3⁺ T-cells in human CCM lesions, serial paraffin-embedded sections of excised CCM lesions from patients 7 and 8 were single and double immunostained by standard immunohistochemistry. Briefly, after antigen retrieval, primary anti-human CD3 antibody (abcam, ab16669, SP7, diluted to a concentration of 1:25), was incubated on tissue sections for 1 hour. The antigen-antibody binding was detected with the secondary antibody (HRP-labeled polymer conjugated goat anti-rabbit IgG, Dako) and DAB+ substrate chromogen (DAKO) system. Primary anti-human CD20 (Dako, M0755, L26, 1:200) antibody was added overnight at 4° C. The antigen-antibody binding was detected with MACH 3 Mouse AP Polymer Detection (Biocare Medical) and Warp Red Chromogen kit (Biocare Medical).

To detect T-cell subtypes, and determine aggregation with B-cells, Applicants performed single stains of anti-CD4 or anti-CD8, and double stains of anti-CD20 with either anti-CD4 or -CD8. Specifically, after antigen retrieval, anti-CD4 (SpringBioscience, M3352, clone SP35; 1:50) or anti-CD8 (SpringBioscience, M3162, clone SP16; 1:50) was added to tissue sections for 1 hour. To detect anti-CD4, tissue sections were incubated with biotinylated anti-rabbit IgG (Vector laboratories; 1:200) for 30 minutes. The antigen-antibody binding was detected by Elite kit (Vector Laboratories) and DAB (DAKO) system. After the second antigen retrieval, anti-CD20 (1:200) was added overnight at 4° C. Anti-CD20 binding was detected with MACH 3 Mouse AP Polymer Detection (Biocare Medical) reagent and a Warp Red Chromogen kit (Biocare Medical). For CD8⁺ T-cell staining, the antigen-antibody binding was detected with Envision+ system (DAKO) and DAB+ chromogen (DAKO). Anti-CD20 (1:200) antibody was used for overnight incubation at 4° C. The antigen-antibody binding was detected with MACH 3 Mouse AP Polymer Detection (Biocare Medical) reagent and a Warp Red Chromogen kit (Biocare Medical). Tonsil was used as a positive control for detecting the presence of B- and T-cells. Isotype controls were used as negative controls for immunostaining.

Immunohistochemical staining of CD138⁺ plasma-cells in CCM lesions for laser captured microdissection (LCM). Fresh frozen tissue from patients 9-10 was immediately frozen in OCT media in the operation room. All the materials that might be in contact with RNA were pretreated with RNase Away (SIGMA), and all solutions were prepared with DEPC-treated water containing 200 units/ml RNase inhibitor (Invitrogen). Ten micron cryo-sections were fixed with 100% acetone for 5 min. Slides were treated with TBST (DAKO) for 2 min, blocked in 0.25% casein serum-free protein block (Dako) for 5 min, and incubated for 10 min with a 1:200 dilution of mouse anti-human CD138 (clone MI15; Dako). Sections were incubated for 10 min with an EnVision® peroxidase-labeled polymer conjugated to anti-mouse antibody (Dako), washed in TBST for 2 min, incubated for 5 min with DAB (Dako), and counterstained with Meyer's hematoxylin solution (Dako) for 15 sec. After dehydration in a series of nuclease-free graded alcohols (HistoGene® Kit, Molecular Devices), the slides were cleared using two changes of xylene, each two minutes long. All incubation steps were performed on ice.

LCM of CD138⁺ plasma-cells from CCM lesions. LCM was conducted on a PixCell II microscope (Molecular Devices, Sunnyvale, Calif.) with CapSure HS caps (Molecular Devices) using a pulse power of 70 mW, a 7.5 μm laser spot diameter, pulse duration of 5 ms and a target voltage of 170 mV. Cell location was visualized by applying 10 μl xylene to sections to reveal the brown DAB color of CD138⁺ plasma-cells. The CD138⁺ plasma-cells were captured and tissue adjacent to the laser-targeted cell was removed with a sterile adhesive-backed paper (Post-it note, 3M, St. Paul, Minn.). The captured cells were immediately stored at −20° C. until completion of LCM.

Cell lysis and cDNA synthesis from RNA in CD138⁺ plasma-cells. Cell lysis was carried out with 21 μl 1× SuperScript III first-strand RT buffer containing 1% NP-40 (Sigma) at 42° C. for 20 min, after which the RT reaction was conducted on the cap surface containing the laser-captured CD138+ plasma-cells. A mixture containing 2 μl 5× first strand buffer, 0.5 μl random hexamers (Invitrogen), 1 μl H₂O and 0.5 μl RNase inhibitor was added, and incubated at 65° C. for 3 min, and then at 25° C. for 3 min. To the reaction, 3 μl 0.1 M dTT, 1.5 μl dNTPs, 0.5 μl RNase inhibitor, and 0.5 μl SuperScript III (Invitrogen) were added, followed by incubation at 50° C. for 60 min, and at 95° C. for 10 min. The product of the RT reaction conducted on the cap was collected by centrifugation.

PCR amplification of the IgGHV and IgGLV. Igγ, Igλ, and Igκ variable regions were amplified by nested PCR. Four microliter aliquots of RT reaction above were used to amplify the IgGHV and IgGLV from laser captured CD138⁺ plasma-cells by 34 cycles of primary PCR (94° C. for 30 sec, 55° C. for 30 sec and 72° C. for 1 min) as described previously (Owens et al., 2003). H-chain primary PCR used all five VH leader forward primers and the CH1 constant-region reverse primer. L-chain primary PCR used all thirteen VL leader forward primers and two constant-region reverse primers, Cκ1 and Cλ1. Nested PCR for L and H chains was conducted under the same conditions as the primary PCR, except with an annealing temperature of 56.5° C. for H chain, five VH Framework 1 forward primers and the constant-region CH2a reverse primer for H-chain amplification and twelve VL Framework 1 forward primers and two constant region reverse primers Cκ1D and Cλ1D for L-chain amplification.

Sequence analysis. Nested PCR products of the entire V region for both H and L chains were cloned into the pGEM®-T Easy Vector Systems (Promega), and plasmid DNA was purified using Wizard® Plus SV Minipreps DNA Purification Systems (Promega). Multiple bacterial clones were randomly selected for sequencing to ensure proper sampling. The V-QUEST program from IMGT was used to identify the closest matching V(D)J germ-line, as well as the CDRs and FWRs. Nucleotide and amino acid mutations in the V-region were identified by alignment with the closest corresponding germ-line using IMGT. Multiple nucleotide changes in a single codon were scored as a single replacement mutation. Related clones were defined by similar CDR3 regions as identified by the junction analysis software provided by IMGT. Sequences that were out of frame or contained mutations that resulted in a non-productive sequence were excluded from analysis. In order to mitigate any errors Applicants have employed two quality control steps which define respective variable chain sequences. These have been used in Applicants' previous analogous publication (Chang et al., 2011) and involve 1. Obtaining each bona fide sequence from at least 2 different picked colonies on the bacterial plate and 2. Confirming that the respective clone differs by three or more nucleotides from other confirmed sequences (Chang et al., 2011). Genealogical trees showing the relationships between plasma-cells were constructed by analysis of the pattern of somatic mutations.

Results

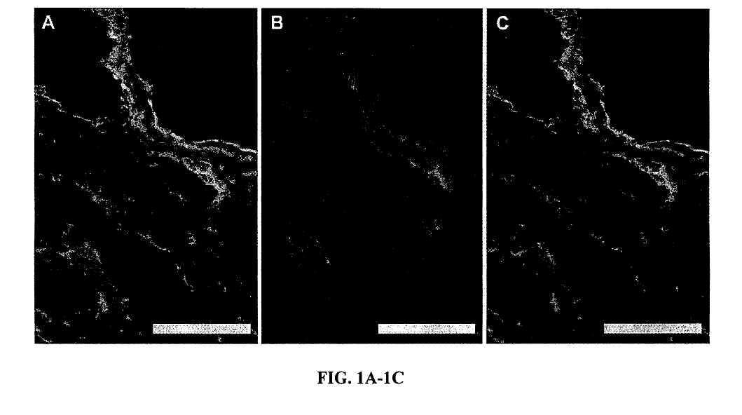

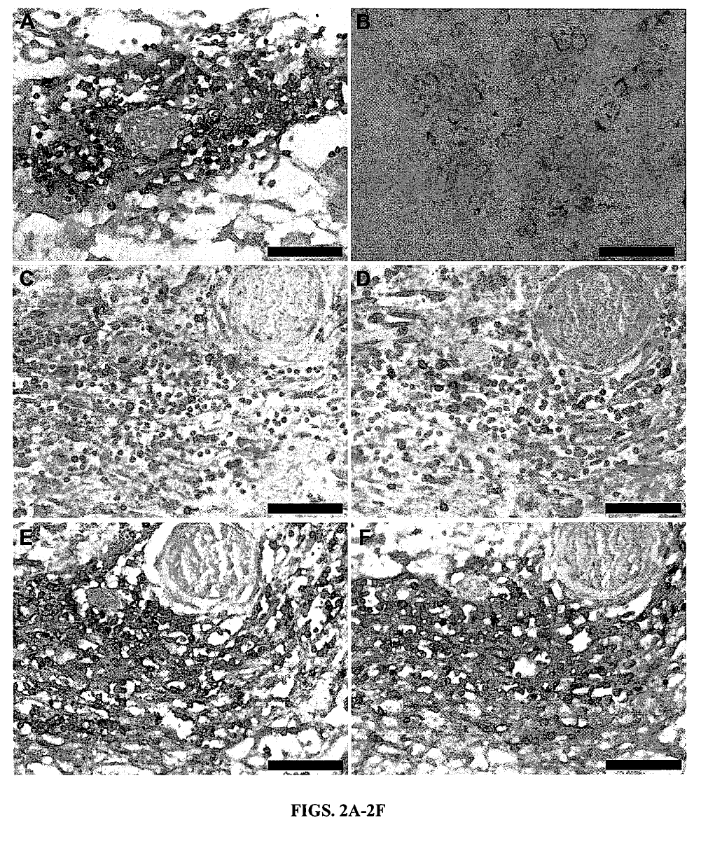

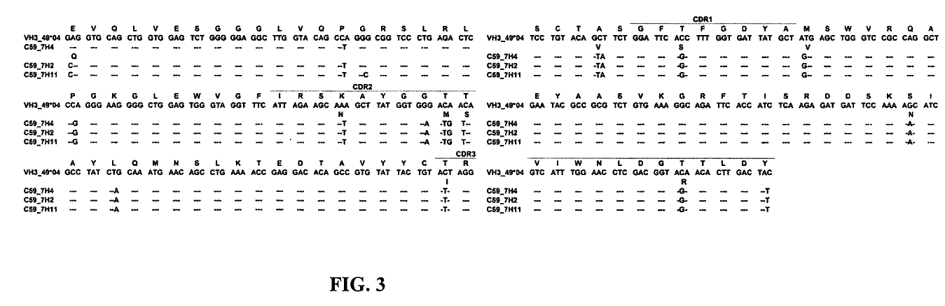

Terminal complement co-localization with IgG expression in human CCM lesions. Although Applicants previously demonstrated IgG deposition in human CCM lesions (Shi et al., 2009), it was not determined whether IgG deposition in human CCM lesions is pathogenic. Here, CCM lesions were assayed for antibody dependent complement activation by co-deposition of IgG and MAC by immunofluorescence. FIGS. 1A, B, and C show the co-localization of IgG and the MAC in the endothelial lining of the caverns within the lesion from patient 1. Four other CCM lesions (from patients 2-5) showed similar results (data not shown). However, a sixth CCM lesion showed no staining for IgG or MACs. Antibody containing immune complexes is therefore common pathological features in most, but not all, CCM lesions.

T-B aggregates in human CCM lesions. To examine whether B- cells and T-cells were in close proximity (which could allow them to undergo cognate interactions) in the CCM lesion, Applicants performed co-stains and single stains of serially sectioned paraffin embedded tissues. T- and B-cells in two different excised human CCM lesions were clustered and co-localized (FIG. 2A). Likewise, most of the plasma-cells in two separate patient lesions were clustered into discrete areas (FIG. 2B). T cell subtypes demonstrated were CD4⁺ (FIG. 2C) and CD8⁺ (FIG. 2D). Co-localization with CD20⁺ B-cells (FIGS. 2E and F) was evident for both sub-types. These data are consistent with CD4⁻ T-cells being readily available to provide help for B-cell class-switching, somatic hypermutation, and differentiation into antibody producing plasma-cells in situ. Likewise, B-cells would also be readily available to activate neighboring T-cells, enabling them to exert their own pathogenic efforts. The spatial organization of clustered B-cells, T-cells, and plasma-cells was remarkably similar to the observed histological structures (T-B aggregates) in the recently described, organ specific, autoimmune disease lupus tubulointerstitial nephritis (Chang et al., 2011).

Characterization of in situ Ig repertoire: Ig clonal restriction and antigen-driven somatic mutation in human CCM lesions. Given that both antibody producing cells and deposited IgG are common in CCM lesions, Applicants next determine whether locally deposited IgG could be produced in situ. If antigen driven B-cell activation and subsequent autoantibody production were occurring in situ, there would likely be clonal relatedness between B-cells and/or plasma-cells within the lesion. To obtain evidence for in situ clonal relatedness, Applicants used LCM coupled to RT-PCR and sequencing to characterize IgG H and L chain variable region repertoires expressed in situ by plasma-cell clusters within the CCM lesions from patients 9 and 10. A single capture of clustered CD138⁺ plasma-cells containing approximately 15 cells were analyzed following respective bulk amplifications in patient 9. Expressed IgG variable regions, and their most closely matching germ-line segments, are provided in Table 1. A total of 4 distinct y and 1 distinct λ sequences were identified. The most commonly occurring gene rearrangement was VH3-49*04 D1-1*01 J4*02 (3 out of the 6 different sequences). The high frequency of sequences corresponding to a single gene rearrangement is consistent with multiple clustered plasma-cells arising from one common progenitor clone that had been antigen receptor activated, proliferated, and given rise to a succession of somatically hypermutated daughter clones.

TABLE 1
Predicted H-chain VDJ and L-chain VJ gene segments
used by IgG expressed in situ in human CCM lesions.

| Patients | Chains | Predicted germline |
|---|---|---|
| 9 | H | VH3-49*04 D1-1*01 J4*02 |
| | | VH3-20*01 D2-2*01 J6*02 |
| | | VH1-2*02/VH1-2*05 D5-12*01 J4*02 |
| | | VH1-69*01/VH1-69D*01 D1-7*01 J3*02 |
| | L (λ) | VL1-44*01 J2*01/J3*01 |
| 10 | H | VH5-51*01 D3-10*01 J4*01/J4*02/J4*03^{a} |
| | | VH1-2*02 D2-21*02 J6*02^{b} |
| | | VH1-69*06 D4-11*01 J4*02/J4*03^{c} |
| | L (K) | VK1-9*01 J4*01^{a} |
| | | VK1-5*01 J2*01/J2*02^{b} |
| | | VK1-33*01/VK1D-33*01 J1*01^{c} |

λ = lambda;
K = kappa.
^{a}Expressed IgG genes found in two different paraffin embedded sections.
^{b}Expressed IgG genes found only in one paraffin embedded section.
^{c}Expressed IgG genes found only in the other paraffin embedded section.

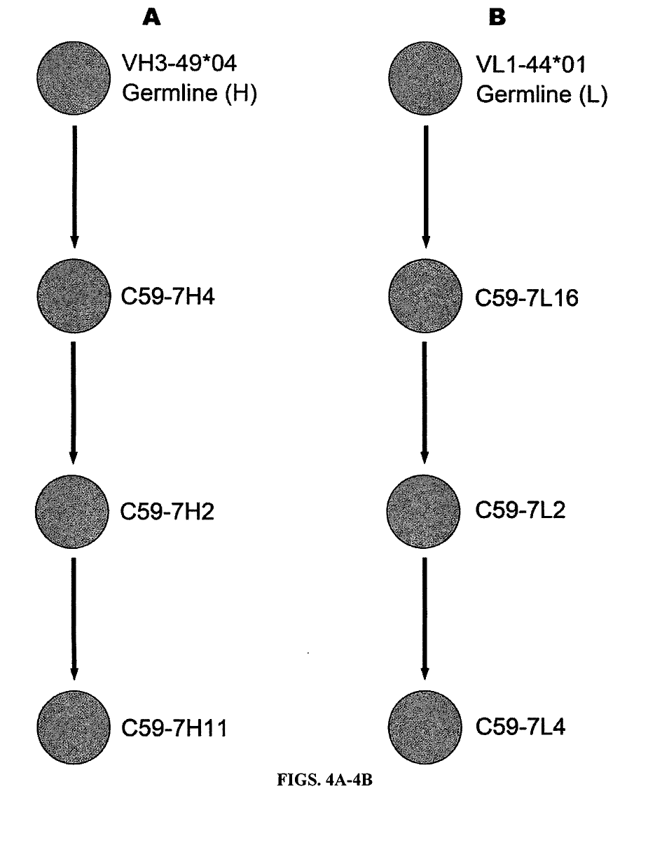

All of the VH3-49*04 D1-1*01 J4*02-matched sequences were heavily mutated compared with the reported germ-line segment, with all containing a similar core of coding and noncoding mutations (FIG. 3). Alignment of the different expressed sequences suggested that cells had sequentially acquired variable region mutations through somatic hypermutation during clonal expansion. The relative genealogy of these mutations is provided in the corresponding clonal tree (FIG. 4A). The clustering of clonally related plasma-cells expressing VH genes with successive mutations is again analogous to that seen in human autoimmune disease lupus tubulointerstitial nephritis (Chang et al., 2011).

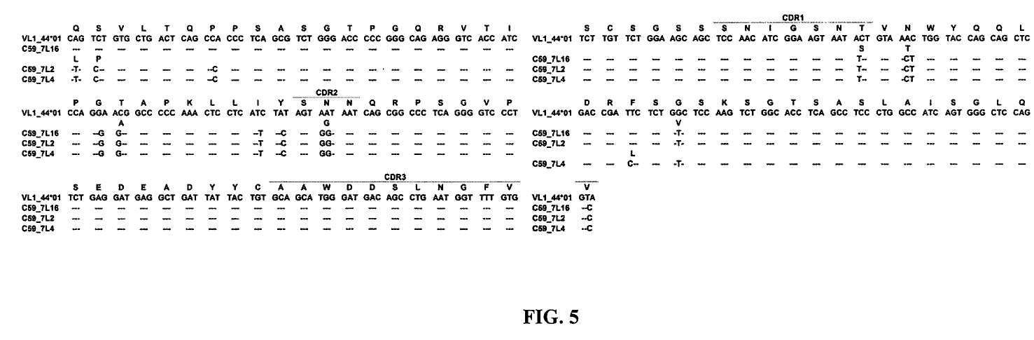

A similar clonal relationship was observed when the λ-chain repertoire of patient 9 was analyzed. All the distinct sequences arose from a single rearrangement (VL1-44*01 J2*01/J3*01) (FIG. 5 and Table 1), suggesting that each of the clustered plasma-cells also arose from a single recombination event/clone. Alignment of the different VL1-44*01 J2*01/J3*01-derived sequences with predicted germ-line sequences indicated that all had accumulated somatic hypermutations (FIG. 5). Assembly of the different VL1-44*01 J2*01/J3*01 sequences into a clonal tree demonstrated a mutational hierarchy, consistent with ongoing clonal expansion and somatic mutation (FIG. 4B).

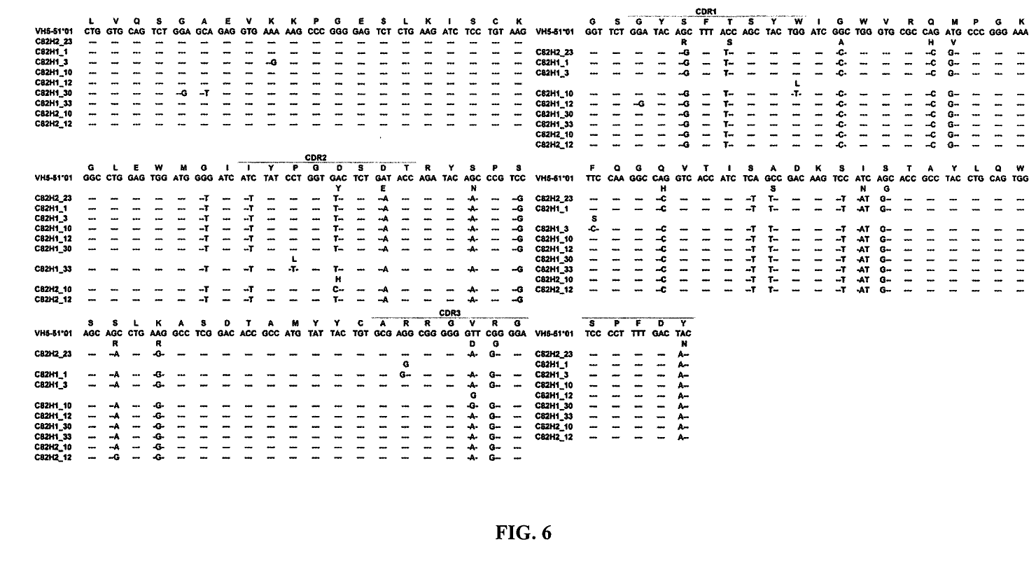

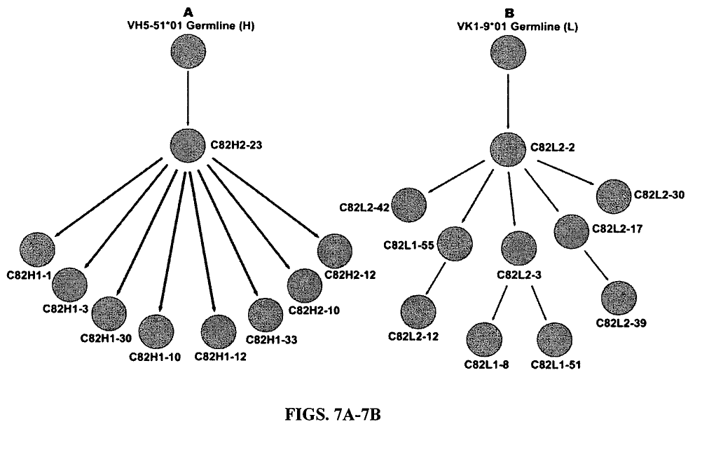

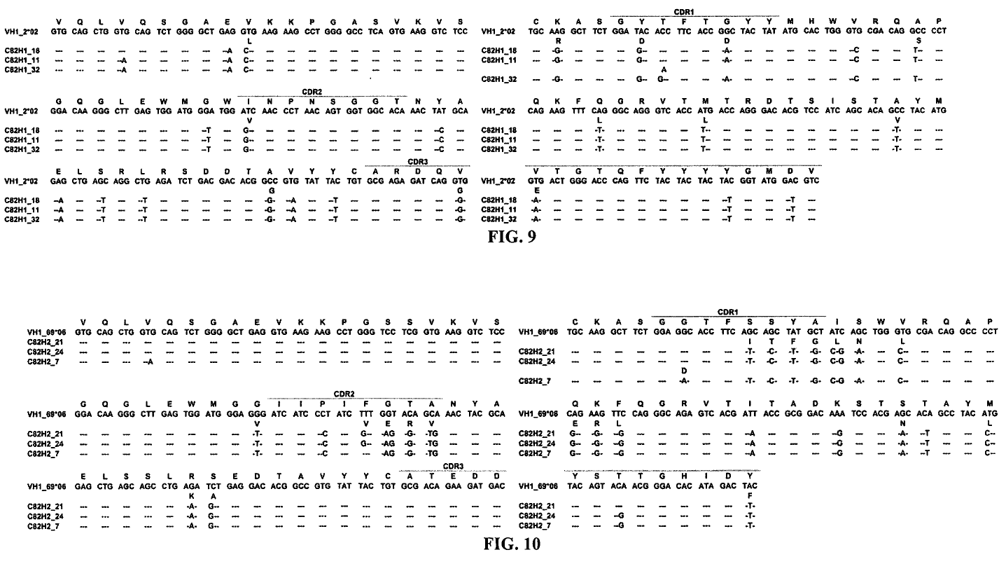

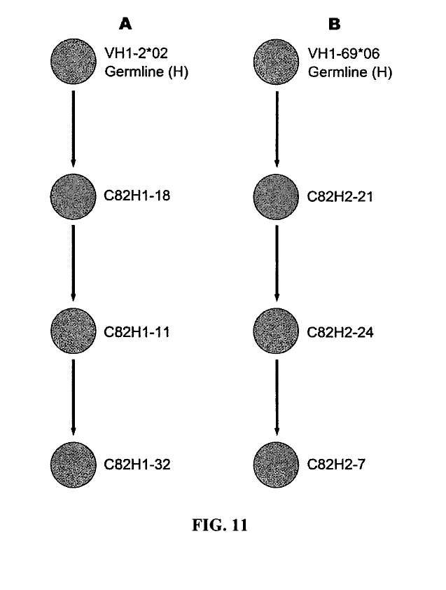

Analysis of the Ig repertoire expressed in the lesion from patient 10 provided further evidence of in situ clonal selection in CCM. A single capture of clustered CD138⁺ plasma-cells containing approximately 50 cells were analyzed following respective bulk amplification in patient 10. Distinct γ and κ sequences are given in Table 1. Within the γ repertoire, three rearrangements were observed more than once. The most common rearrangement was VH5-51*01 D3-10*01 J4*01/J4*02/J4*03 (FIG. 6). Alignment of amplified variable regions sequences with the predicted germ-line sequence indicated extensive somatic hypermutation (FIG. 6). Expressed variable region sequences could therefore be assembled into a simple clonal tree, suggestive of ongoing somatic mutation (FIG. 7A). The other two gene rearrangements observed in that particular lesion were VH1-2*02 D2-21*02 J6*02 and VH1-69*06 D4-11*01 J4*02/J4*03. Similarly, alignment of these cDNA fragments with the predicted germ-line sequences demonstrated that the sequences had undergone extensive somatic hypermutation (FIGS. 9 and 10). The sequences could therefore also be assembled into simple clonal trees (FIG. 11). The evidence for somatic hypermutation by progressively related subgroups of clones is far more strengthened by the minor sequences of patient 10 (FIGS. 9 and 10) and patient 9 (FIGS. 3 and 4A).

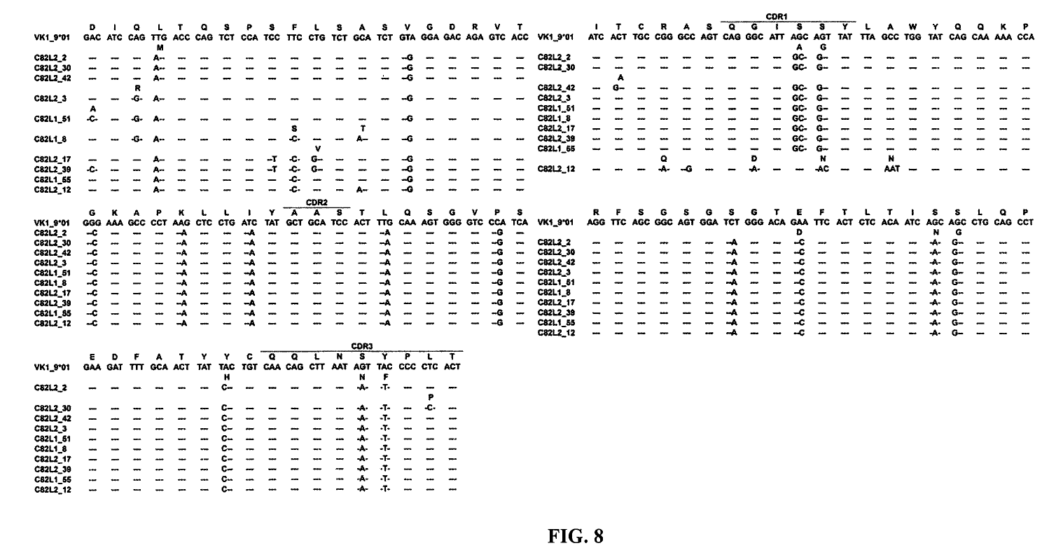

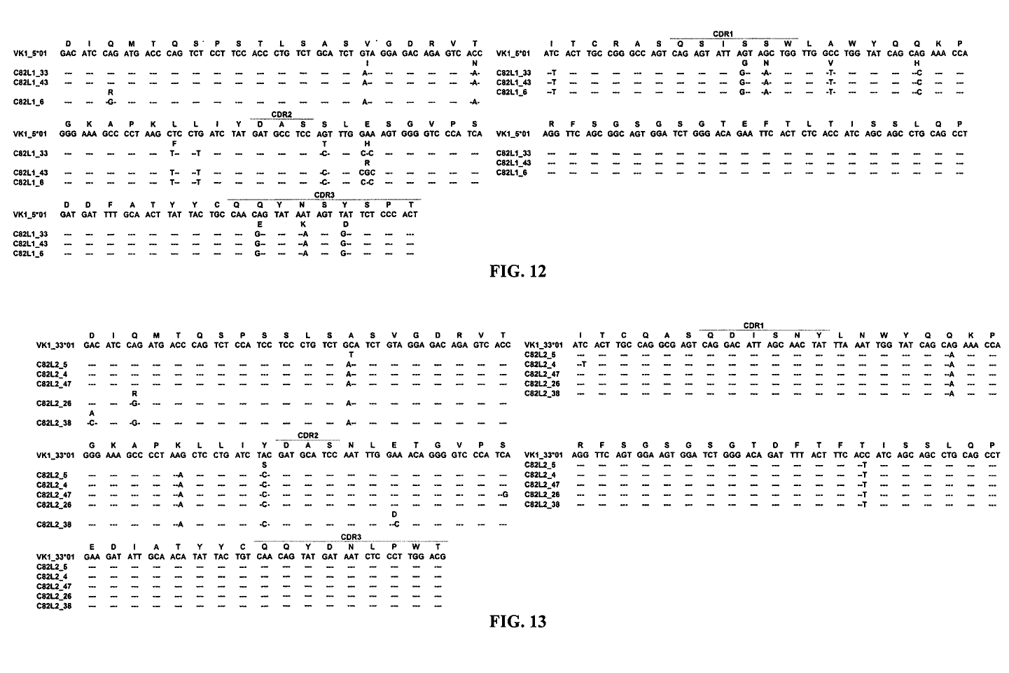

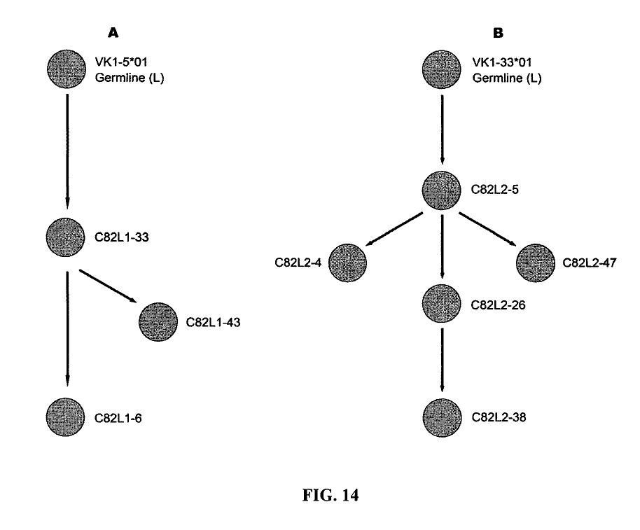

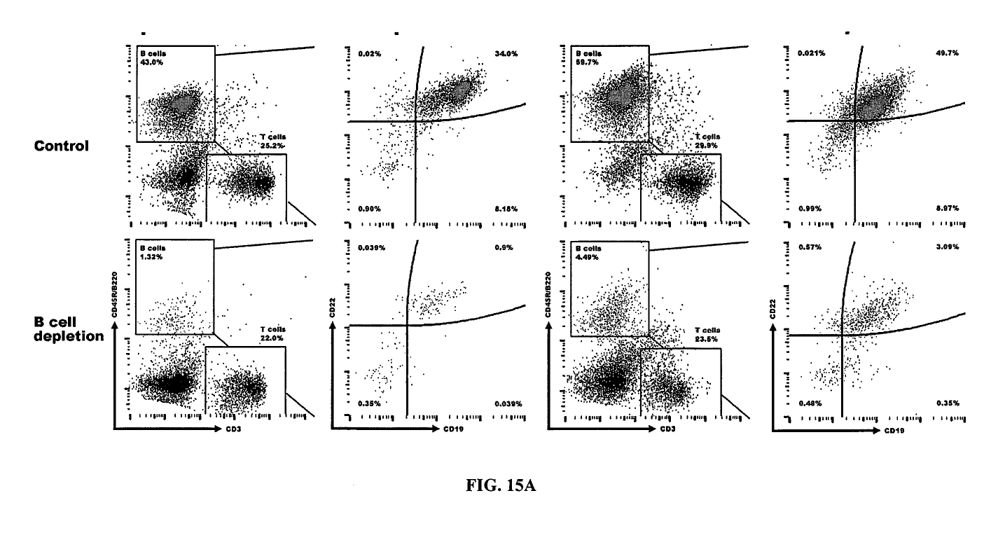

Clonal relatedness was also demonstrated for the L chain variable region repertoire expressed in the lesion of patient 10. Three rearrangements were found more than once (VK1-9*01 J4*01, VK1-5*01 J2*01/J2*02, and Vκ1-33*01NK1D-33*01 J1*01). Alignment of the sequences matching to VK1-9*01 J4*01 indicated extensive somatic mutation (FIG. 8). Once again, these clones could be assembled into a simple clonal tree, suggestive of in situ selection (FIG. 7B). Likewise, alignment of the less frequently observed sequences with their respective germ-line sequences (VK1-5*01 J2*01/J2*02 (FIG. 12) and VK1-33*01NK1D-33*01 J1*01 (FIG. 13)) and the construction of clonal trees indicated successive rounds of B-cell proliferation and somatic hypermutation (FIG. 14).

Discussion

In this study, further evidence was generated in support of Applicants' hypothesis of in situ antigen driven B-cell clonal expansion, antibody production and the formation of local antibody and complement containing immune complexes within CCM lesions. Although there is a presence of IgG in human CCM lesions (Shi et al., 2009), it remained unclear whether IgG deposition in CCM is pathogenic. Herein, for the first time, Applicants provide evidence for the formation of IgG containing immune complexes, which are likely mediators of local inflammation. Immune complexes were found in the vicinity of endothelial cells suggesting that either they, or the surrounding matrix, are targeted by pathogenic antibodies. Membrane attack complexes (MACs) which are terminal products of complement activation cascade, demonstrated herein may be playing an active role in endothelial cell dysfunction via the formation of transmembrane channels and induction of apoptotic cell death (Peitsch and Tschopp, 1991). Interestingly, MAC has been implicated as a pathogenic mediator in numerous vascular diseases, such as anti-neutrophil cytoplasmic autoantibody associated vasculitis, where it locates to glomerular capillaries (Xing et al., 2009), in terbinafine-induced dermatomyositis, where it is found in the cutaneous microvasculature (Magro et al., 2008), and diabetes mellitus and porphyria cutanea tarda (Vasil and Magro, 2007). MACs have also been demonstrated in cerebral aneurysms, where they are involved in aneurysm wall degeneration and rupture (Tulamo et al., 2006). Thus, co-localization of IgG and MAC in five of the six CCM lesions supports the involvement of immune complex formation and IgG mediated pathogenesis in the majority of lesions. However, the cases in this study were too few to glean any correlative clinical differences between the inflammatory and non-inflammatory CCMs. In addition, our previous study including 23 cases had not shown a relationship of CCM lesion immune cell infiltration with recent bleeding, lesion growth or other aspect of recent clinical activity (Shi et al., 2009). Hence the immune and inflammatory response is an inherent feature of the CCM phenotype, rather than a reaction to recent bleeding or lesion growth. This pathogenic pathway is analogous to antibody- and complement-mediated central nervous system damage, which are the dominant mechanisms in the pathogenesis of multiple sclerosis, widely considered as an antibody mediated autoimmune disease (Breij et al., 2008, Scolding et al., 1989).

The autoimmune phenomenon in CCMs is attributed to the unique neo-antigenic milieu in CCM lesions, including the characteristic vascular phenotype with the sequestration, within caverns, of thrombi at various stages of organization, leaky blood-brain barriers, and the chronic deposition of iron and blood degradation products. Macrophages have long been recognized to infiltrate CCM lesions, especially in reaction to acute bleeding (Vymazal et al., 1996). Macrophages were also noted in histologic analysis of the first CCM lesions produced in the transgenic mice (Plummer et al., 2004), and subsequently confirmed in Applicants' and other transgenic mice with CCMs (Chan et al., 2011, McDonald et al., 2011). Also, macrophages are all frequently identified in the vascular wall of cerebral arteriovenous malformations (AVM) tissues (Chen et al., 2008), and macrophage infiltration has been well documented in both human and animal intracranial aneurysms (Chyatte et al., 1999, Kanematsu et al., 2011). The role of macrophages in CCM lesions is not known. They may be important for the clearance of apoptotic debri, but may also mediate pathogenesis by providing the necessary antigen presentation function to activate infiltrating autoreactive T-cells in CCM lesions. Interestingly, B- and T-lymphocytes are rarely observed in cerebral AVM specimens (Chen et al., 2008), indicating that B- and T-lymphocytes are not involved in the pathogenesis of all lesions occurring at the same anatomical location, and that their involvement is restricted to specific lesional milieu of CCM.

B- and T-cells were found to aggregate in a pattern typical of many previously documented organ specific antibody mediated autoimmune diseases. CD4⁺ T-cells are essential for inducing somatic hypermutation and class-switching in B-cells. Moreover, clusters of plasma-cells were observed in the majority of CCM lesions, suggesting a pathological role for local antibody secreting cells and their secreting antibodies. A likely source of the locally deposited antibodies are the resident plasma-cells that may have differentiated from in situ B cells following interactions with CD4⁺ T-cells. These plasma cells expressed somatically hypermutated, clonally related and IgG class-switched antibodies. A combination of laser microdissection and sensitive PCR technologies were used to analyze the IgG variable regions expressed by plasma-cells within the CCM lesions to test the hypothesis that B cell clonal expansion and differentiation into plasma-cells is occurring in situ, and is driven by specific antigens. The analyses of the IgG H and L chain V region repertoires expressed by clustered plasma-cells in CCM lesions from two patients enabled the generation of clonal trees, consistent with in situ somatic hypermutation and affinity maturation (Odegard and Schatz, 2006). The same translated amino acid motifs in the variable regions of clones expressed by different patients were not observed. Inter-patient diversity of IgG repertoires in CCM suggests that target epitopes vary between lesions. In contrast to the distinct use of the V regions of Ig gene by plasma-cells from different patients, the same Ig sequences were observed in different areas of the same lesion from one of the patients (patient 10, Table 1). This indicates that B cells in different areas of the same lesion undergo antigen driven expansion in response to a common trigger, and that a restricted antibody repertoires emerges with dominant clonotypes for each lesion. Alternatively, there may be migration of antigen-specific B-cells or plasma-cells from one anatomical site to another. The data taken together suggest complexity in the B-cell and plasma-cell repertoire potentially reflective of CCM disease heterogeneity or variability of disease duration in CCMs. This characteristic of clonal expansion of B- and plasma-cells in CCM lesions is analogous to that observed for human multiple sclerosis (Owens et al., 2003), lupus nephritis (Chang et al., 2011), and Kawasaki Disease (Rowley et al., 2005), consistent with the adaptive autoimmune inflammatory component to CCM pathogenesis.

Although progeny of B-cells within clonal trees have undergone antigen driven selection in CCM lesions, the identification of putative antigens in CCMs remains unknown. Evidence for in situ antigen driven clonal expansion could be further supported by demonstration of activation-induced cytidine deaminase expression (AID) within the lesion. However, definitive proof can only be demonstrated by the generation of sequentially mutated antibodies and assaying their reactivity with their characterized driving antigen. Thus, generation of monoclonal antibodies with variable regions expressed by antibody secreting cells found in CCM lesions would provide the necessary tools for localizing and characterizing putative antigenic triggers perpetuating this defined immune response. The production of these antibodies from in situ clonally expanded plasma-cells is in progress, based on immunoglobulin variable region sequences herein characterized. B cell depletion therapy provides proof of principle in therapeutic modification.

In summary, Applicants have demonstrated the presence of an active in situ organ specific adaptive humoral immune response in CCM, and the formation of local antibody containing immune complexes. Moreover, the cells necessary for the generation of a T-cell dependent class-switched antibody response can be found within the same vicinity. B-cells have undergone in situ clonal expansion, indicating antigenic stimulation in CCMs. These data provide further compelling evidence for autoimmunity within the CCM lesion being a contributory factor to CCM. Identification of targeted antigens and the molecular and cellular interactions that prompt the autoimmune response should provide further insights into the pathobiology of CCMs, enable the generation of biomarkers, and identify targets for therapies in CCM.

### Example 2

### B-Cell Depletion in Ccm3+/− Mice

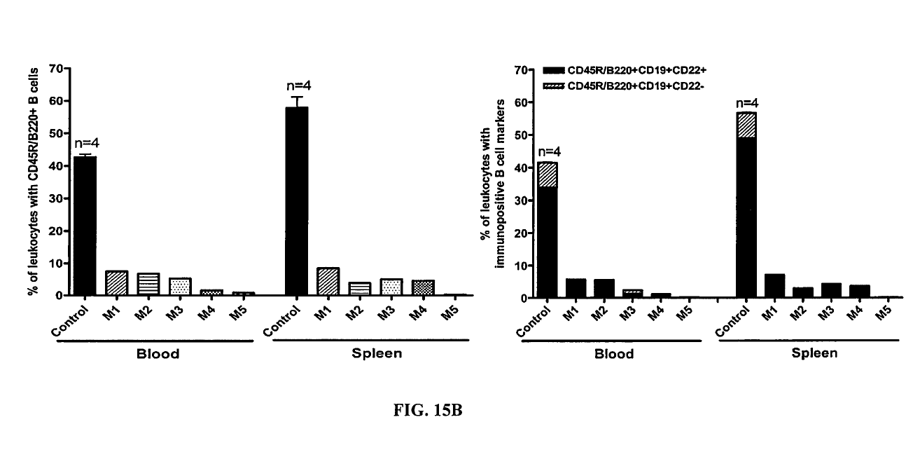

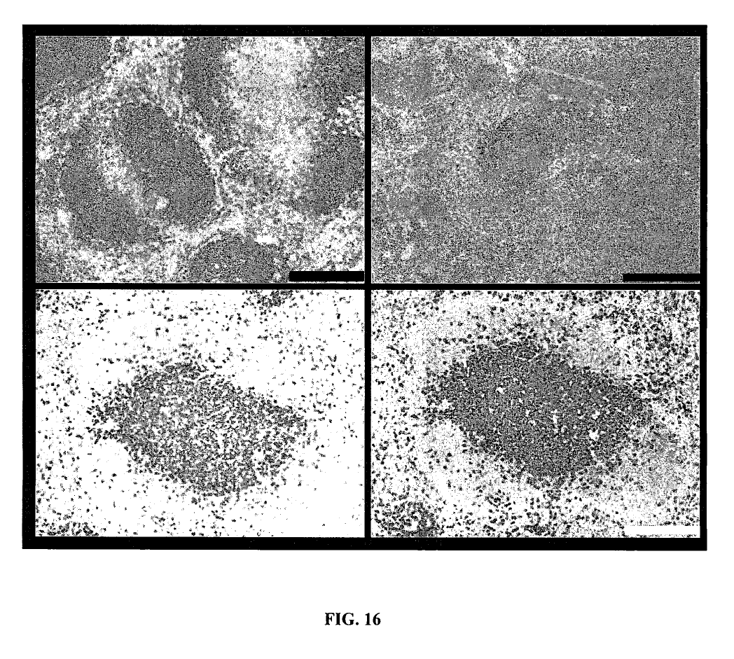

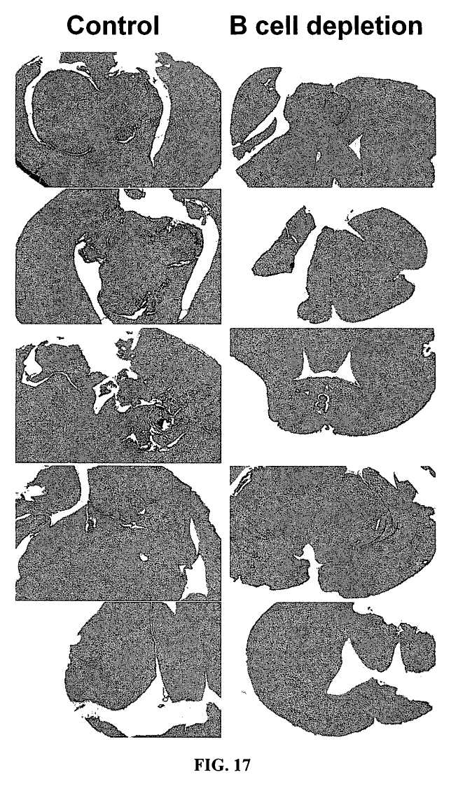

To test the effects of B-cell depletion in CCM mice, Ccm3+/−Trp53−/− mice were used as a CCM model (100% penetrance with severe lesion burden). Anti-BR3 antibody was used for B cell depletion. This antibody works to deplete B cells and to block B cell activating factor (BAFF or BLys)-mediated B-cell survival. (BR3: BLys receptor 3). The mice were subjected to intraperitoneal injections once a week at a 5 mg/kg/week for a total of 12-15 weeks. These antibodies combine two mechanisms of action and show increased in vivo B-cell reduction in some subsets compared with anti-CD20 mAb-mediated B-cell depletion or BAFF-dependent survival blockade alone. As shown in FIG. 15A-B, mice treated with the B-cell depletion therapy showed a reduction in the number of B cells in the blood from the spleen of the mice. This was confirmed in five out of five mice. FIG. 16 further confirms these results by immunostaining of histology sections from the spleen of the CCM mice. A reduction of B cells (top panel) but not T cells (bottom panel) was seen in B-cell depleted mice (right panel), but not control mice (left panel). FIG. 17 shows representative lesions from control and B-cell depleted mice. As shown in FIG. 17, B-cell depleted mice exhibit a significant reduction in the size and number of CCM lesions.

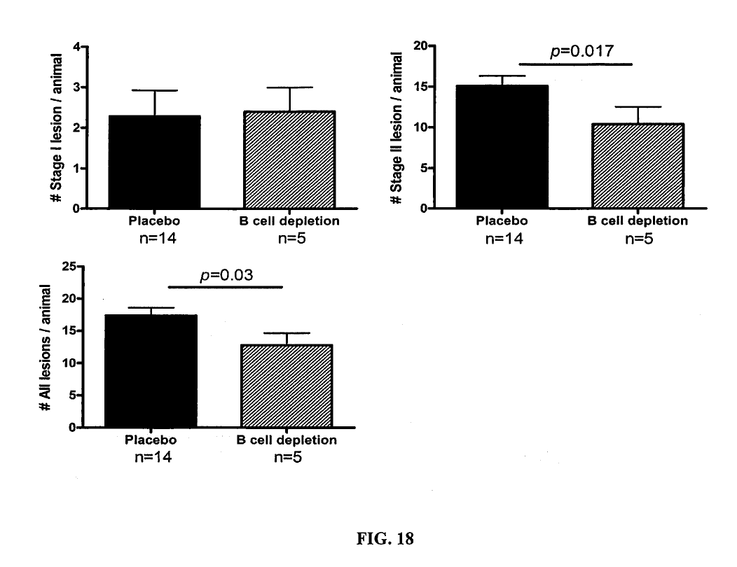

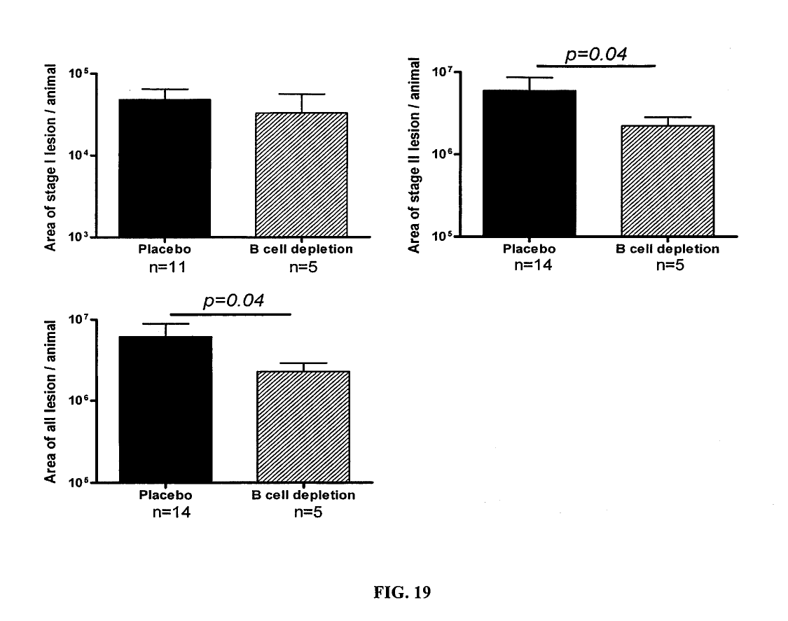

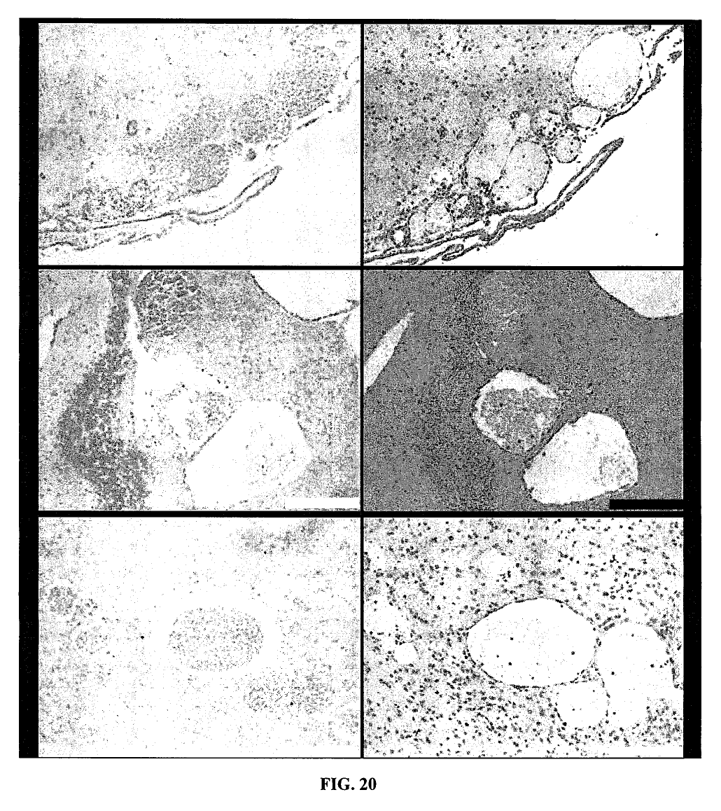

To quantify the B-cell depletion results, lesion burden was assessed. 1 mm serial coronal slices of each mouse brain were systemically assessed for capilllary ectasia (Stage I) pre-lesions, and multicavernous (Stage 2) mature CCM lesions. The number and areas of Stage 1 and 2 lesions were assessed. Stage 1 CCM lesions are characterized as having dilated capillaries having the width at least 25 red blood cells and not joined to any other lesion (isolated caverns). Stage 2 lesions are characterized by multicavernous structures composed of the confluence of two or more caverns (See McDonald et al. A novel mouse model of cerebral cavernous malformations based on the two-hit mutation hypothesis recapitulates the human disease. Hum Mol Genet. 2011; 20: 211-22 for further information). As shown in FIGS. 18-19, the number (FIG. 18) and area (FIG. 19) of Stage II CCMs were significantly reduced in B-cell depleted mice. To further investigate these results, the histology sections of the lesions of Ccm3+/−Trp53−/− mice treated with anti-BR3 antibody were analyzed by immunohistochemistry for B cells. No B cells were detected in the lesions of these mice, indicating that B-cells were depleted in the CCM lesions of these mice (FIG. 20).

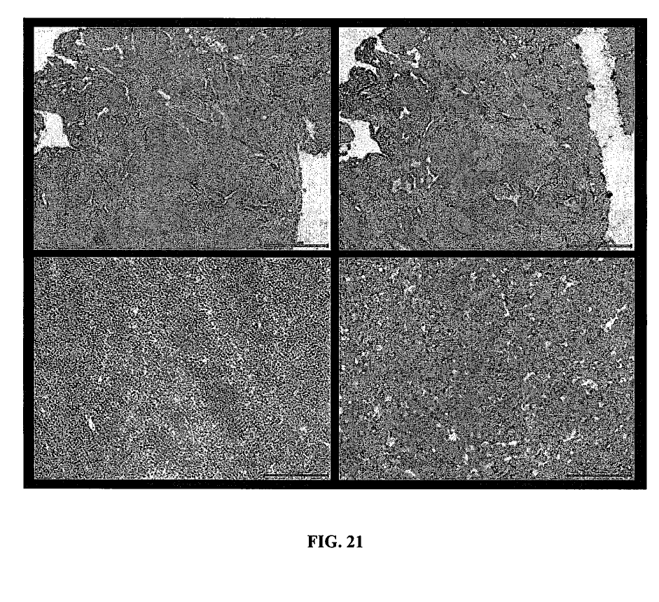

As shown in FIG. 21, extra-cranial tumors were found in 3 out of 5 B-cell depleted Ccm3+/−Trp53−/− mice. No tumors were detected any tumors in 10 out of 10 B-cell deleted Ccm3+/− mice, indicating Trp53−/− background increase the risk of tumors with B cell depletion.

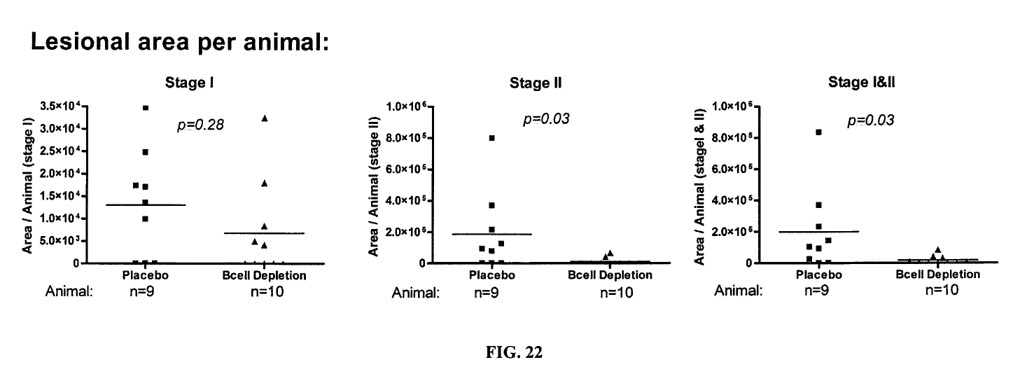

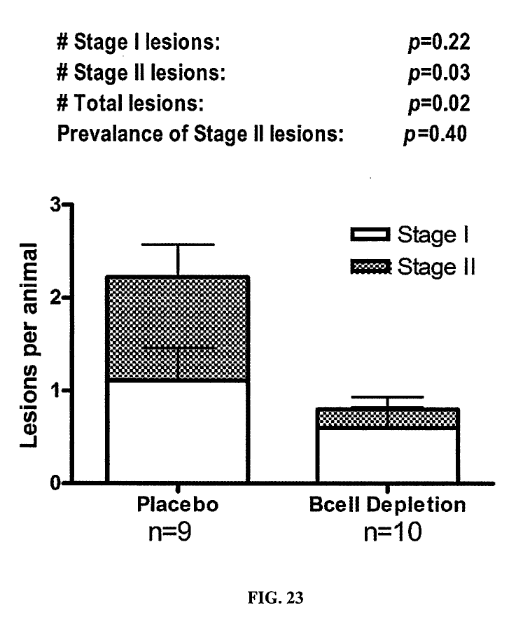

Applicants have further tested the same B cell depletion therapy in heterozygous Ccm3+/− mice without sensitization by p53 loss. These mice have a much lower burden of CCM lesions (milder disease), and are not prone to forming tumors, hence more closely recapitulating the human disease. Indeed, as with the proof of concept observations in the more aggressive Ccm3+/−Trp53−/− mice, we also demonstrate significant decrease in CCM lesion burden in the Ccm3+/− mice, particularly the more mature clinically significant stage 2 lesions. The prevalence of CCM lesions was significantly lower in B cell depleted mice, and these also harbored significantly smaller lesions. This further confirms the benefit of B cell depletion, regardless of p53 sensitization. None of the mice in these non-sensitized mice developed tumors. This data is shown in FIGS. 22-23.

In conclusion, these studies represent the first report of therapeutic benefit of B-cell depleted therapy in the development of mature CCM lesions. This provides proof of principle that B cells are a critical step in CCM lesion genesis and maturation.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Bergametti, et al., Am. J. Hum. Genet. 76, 42-51, 2005.
Bradley, Radiology 189, 15-26, 1993.
Breij, et al., Ann. Neurol. 63, 16-25, 2008.
Chan, et al., J. Clin. Invest. 121, 1871-1881, 2011.
Chang, et al., J. Immunol. 186, 1849-1860, 2011.
Chen, et al., Neurosurgery 62, 1340-1349; discussion 1349-1350, 2008.
Chyatte, et al., Neurosurgery 45, 1137-1146; discussion 1146-1137, 1999.
Denier, et al., Ann. Neurol. 60, 550-556, 2006.
Denier, et al., Am. J. Hum. Genet. 74, 326-337, 2004.
Gault, et al., Neurosurgery 55, 1-16; discussion 16-17, 2004.
Gault, et al., Neurosurgery 59, 1278-1284; discussion 1284-1275, 2006.
Guclu, et al., Stroke 36, 2479-2480, 2005.
Kanematsu, et al., Stroke 42, 173-178, 2011.
Laberge-le Couteulx, et al., Nat. Genet. 23, 189-193, 1999.
Liguori, et al., Am. J. Hum. Genet. 73, 1459-1464, 2003.
Magro, et al., J. Cutan. Pathol. 35, 74-81, 2008.
Maraire & Awad, Neurosurgery 37, 591-605, 1995.
McDonald, et al., Hum. Mol. Genet. 20, 211-222, 2011.
McDonald, et al., Stroke 43, 571-574, 2012.
Odegard & Schatz, Nat. Rev. Immunol. 6, 573-583, 2006.
Otten, et al., Neurochirurgie 35, 82-83, 128-131, 1989.
Owens, et al., J. Immunol. 171, 2725-2733, 2003.
Peitsch & Tschopp, Curr. Opin. Cell Biol. 3, 710-716, 1991.
Plummer, et al., Am. J. Pathol. 165, 1509-1518, 2004.
Revencu & Vikkula, J. Med. Genet. 43, 716-721, 2006.
Rigamonti, et al., J. Neurosurg. 67, 518-524, 1987.
Robinson, et al., J. Neurosurg. 75, 709-714, 1991.
Robinson, et al., Neurosurgery 33, 547-554; discussion 554-545, 1993.
Rowley, et al., J. Immunol. 175, 8386-8391, 2005.
Sahoo, et al., Hum. Mol. Genet. 8, 2325-2333, 1999.
Scolding, et al., Nature 339, 620-622, 1989.
Shi, et al., Stroke 40, 1659-1665, 2009.
Shi, et al., J. Neurosurg. 107, 1023-1026, 2007.
Steiger, et al., Neurosurgery 21, 879-884, 1987.
Tu, et al., J. Neurosurg. 103, 903-909, 2005.
Tulamo, et al., Neurosurgery 59, 1069-1076; discussion 1076-1067, 2006.
Vasil & Magro, J. Am. Acad. Dermatol. 56, 96-104, 2007.
Vymazal, et al., Magn. Reson. Med. 35, 56-61, 1996.
Wong, et al., Neurosurgery 46, 1454-1459, 2000.
Xing, et al., J. Clin. Immunol. 29, 282-291, 2009.

1. A method for treating cerebral cavernous malformations (CCMs) in a patient determined to be in need thereof comprising administering a therapeutically effective amount of a B-cell immunomodulation therapy to the patient.

2. The method of claim 1, wherein the B-cell immunomodulation therapy is a B-cell depletion therapy.

3. The method of claim 1 or 2, wherein the B-cell immunomodulation therapy comprises an antibody.

4. The method of claim 3, wherein the antibody is a human antibody, humanized antibody, recombinant antibody, chimeric antibody, an antibody derivative, a veneered antibody, a diabody, a monoclonal antibody, or a polyclonal antibody.

5. The method of any one of claims 1-4, wherein the antibody is a human antibody.

6. The method of any one of claims 1-5, wherein the antibody comprises one or more of anti-CD20, anti-BLyS, anti-CD19, anti-CD22, and Abatacept.

7. The method of any one of claims 1-5, wherein the B-cell immunomodulation therapy comprises one or more of belimumab, rituximab, ocrelizumab, ofatumumab, MDX-1342, epratuzumab, and atacicept.

8. The method of any one of claims 1-7, wherein the B-cell immunomodulation therapy reduces or inhibits B-cell activation and/or survival.

9. The method of claim 8, wherein the B-cell immunomodulation therapy comprises a B-lymphocyte stimulator (BLyS)-specific inhibitor.

10. The method of claim 8, wherein the B-cell immunomodulation therapy comprises atacicept or belimumab, or both.

11. The method of claim 10, wherein the B-cell immunomodulation therapy comprises belimumab.

12. The method of any one of claims 1-11, wherein the method is for treating Stage II CCMs.

13. The method of any one of claims 1-12, wherein the CCM is sporadic.

14. The method of any one of claims 1-12, wherein the CCM is familial.

15. The method of any one of claims 1-14, wherein the method further comprises surgical resection of the CCM.

16. The method of any one of claims 1-15, wherein the patient has been previously treated for CCM with radiosurgery or surgical resection.

17. The method of any one of claims 1-15, wherein the method further comprises radiosurgery.

18. The method of any one of claims 1-17, wherein the method further comprises administration of an additional immunomodulating agent.

19. The method of claim 18, wherein the additional immunomodulating agent is administered concomitantly.

20. The method of claim 18 or 19, wherein the additional immunomodulating agent is a co-stimulation inhibitor.

21. The method of any one of claims 18-20, wherein the additional immunomodulating agent is Abatacept.

22. The method of any one of claims 1-21, wherein the method further comprises administration of a Rho-kinase (ROCK) inhibitor.

23. The method of claim 22, wherein the Rho-kinase inhibitor is fasudil.

24. The method of any one of claims 1-23, wherein the method further comprises administration of a HMG-CoA reductase inhibitor.

25. The method of claim 24, wherein the HMG-CoA reductase inhibitor is a statin.

26. The method of any one of claims 1-25, wherein the B-cell immunomodulation therapy is administered parenterally.

27. The method of any one of claims 1-26, wherein the B-cell immunomodulation therapy is administered intravenously.

28. The method of any one of claims 1-27, wherein the method further comprises monitoring the patient for B-cell immunomodulation.

29. The method of claim 28, wherein the patient is monitored by magnetic resonance imaging (MRI).

30. The method of claim 28 or 29, wherein the blood of the patient is assayed for B-cell immunomodulation.

31. A pharmaceutical composition comprising a B-cell depleting agent and a ROCK inhibitor.

32. The pharmaceutical composition of claim 31, wherein the B-cell immunomodulation therapy comprises an antibody.

33. The pharmaceutical composition of claim 32, wherein the antibody is a human antibody, humanized antibody, recombinant antibody, chimeric antibody, an antibody derivative, a veneered antibody, a diabody, a monoclonal antibody, or a polyclonal antibody.

34. The pharmaceutical composition of any one of claims 32-33, wherein the antibody is a human antibody.

35. The pharmaceutical composition of any one of claims 32-34, wherein the antibody comprises one or more of anti-CD20, anti-BLyS, anti-CD19, anti-CD22, and Abatacept.

36. The pharmaceutical composition of any one of claims 31-35, wherein the B-cell immunomodulation therapy comprises one or more of belimumab, rituximab, ocrelizumab, ofatumumab, MDX-1342, epratuzumab, and atacicept.

37. The pharmaceutical composition of any one of claims 31-36, wherein the B-cell immunomodulation therapy reduces or inhibits B-cell activation and/or survival.

38. The pharmaceutical composition of claim 37, wherein the B-cell immunomodulation therapy comprises a B-lymphocyte stimulator (BLyS)-specific inhibitor.

39. The pharmaceutical composition of claim 37, wherein the B-cell immunomodulation therapy comprises one or both of atacicept and belimumab.

40. The pharmaceutical composition of any one of claims 31-39, wherein the ROCK inhibitor is fasudil.

41. The pharmaceutical composition of any one of claims 31-40, wherein the composition further comprises a HMG-CoA reductase inhibitor.

42. The pharmaceutical composition of claim 41, wherein the HMG-CoA reductase inhibitor is a statin.


SEQUENCE LISTING (55 sequences)

SEQ ID NO: 1 - protein, 112 aa, Artificial Sequence
  Synthetic Peptide
EVQLVESGGGLVQPGRSLRLSCTASGFTFGDYAMSWVRQAPGKGLEWVGFIRSKAYGGTTEYAASVKGRFTISRDDSKSIAYLQMNSLKTEDTAVYYCTRVIWNLDGTTLDY

SEQ ID NO: 2 - DNA, 336 bp, Artificial Sequence
  Synthetic Primer
gaggtgcagctggtggagtctgggggaggcttggtacagccagggcggtccctgagactctcctgtacagcttctggattcacctttggtgattatgctatgagctgggtccgccaggctccagggaaggggctggagtgggtaggtttcattagaagcaaagcttatggtgggacaacagaatacgccgcgtctgtgaaaggcagattcaccatctcaagagatgattccaaaagcatcgcctatctgcaaatgaacagcctgaaaaccgaggacacagccgtgtattactgtactagggtcatttggaacctcgacggtacaacacttgactac

SEQ ID NO: 3 - DNA, 336 bp, Artificial Sequence
  Synthetic Primer
gaggtgcagctggtggagtctgggggaggcttggtacagcctgggcggtccctgagactctcctgtacagtatctggattcagctttggtgattatgctgtgagctgggtccgccaggctccggggaaggggctggagtgggtaggtttcattagaagcaatgcttatggtggaatgtcagaatacgccgcgtctgtgaaaggcagattcaccatctcaagagatgattccaaaaacatcgcctatctacaaatgaacagcctgaaaaccgaggacacagccgtgtattactgtattagggtcatttggaacctcgacggtacaagacttgactat

SEQ ID NO: 4 - DNA, 336 bp, Artificial Sequence
  Synthetic Primer
caggtgcagctggtggagtctgggggaggcttggtacagcctgggcggtccctgagactctcctgtacagtatctggattcagctttggtgattatgctgtgagctgggtccgccaggctccggggaaggggctggagtgggtaggtttcattagaagcaatgcttatggtggaatgtcagaatacgccgcgtctgtgaaaggcagattcaccatctcaagagatgattccaaaaacatcgcctatctacaaatgaacagcctgaaaaccgaggacacagccgtgtattactgtattagggtcatttggaacctcgacggtacaagacttgactat

SEQ ID NO: 5 - DNA, 336 bp, Artificial Sequence
  Synthetic Primer
caggtgcagctggtggagtctgggggaggcttggtacagcctggccggtccctgagactctcctgtacagtatctggattcagctttggtgattatgctgtgagctgggtccgccaggctccggggaaggggctggagtgggtaggtttcattagaagcaatgcttatggtggaatgtcagaatacgccgcgtctgtgaaaggcagattcaccatctcaagagatgattccaaaaacatcgcctatctacaaatgaacagcctgaaaaccgaggacacagccgtgtattactgtattagggtcatttggaacctcgacggtacaagacttgactat

SEQ ID NO: 6 - protein, 101 aa, Artificial Sequence
  Synthetic Peptide
QSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNGFVV

SEQ ID NO: 7 - DNA, 303 bp, Artificial Sequence
  Synthetic Primer
cagtctgtgctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgagcatgaggctgattattactgtgcagcatgggatgacagcctgaatggttttgtggta

SEQ ID NO: 8 - DNA, 303 bp, Artificial Sequence
  Synthetic Primer
cagtctgtgctgactcagccaccctcagcgtctgggacccccgggcagagggtcaccatctcttgttctggaagcagctccaacatcggaagtaattctgtaacttggtaccagcagctcccaggggcggcccccaaactcctcatttacagtggtaatcagcggccctcaggggtccctgaccgattctctgtctccaagtctggcacctcagcctccctggccatcagtgggctccagtctgagcatgaggctgattattactgtgcagcatgggatgacagcctgaatggttttgtggtc

SEQ ID NO: 9 - DNA, 303 bp, Artificial Sequence
  Synthetic Primer
ctgcctgtgctgactcagcccccctcagcgtctgggacccccgggcagagggtcaccatctcttgttctggaagcagctccaacatcggaagtaattctgtaacttggtaccagcagctcccaggggcggcccccaaactcctcatttacagtggtaatcagcggccctcaggggtccctgaccgattctctgtctccaagtctggcacctcagcctccctggccatcagtgggctccagtctgagcatgaggctgattattactgtgcagcatgggatgacagcctgaatggttttgtggtc

SEQ ID NO: 10 - DNA, 303 bp, Artificial Sequence
  Synthetic Primer
ctgcctgtgctgactcagcccccctcagcgtctgggacccccgggcagagggtcaccatctcttgttctggaagcagctccaacatcggaagtaattctgtaacttggtaccagcagctcccaggggcggcccccaaactcctcatttacagtggtaatcagcggccctcaggggtccctgaccgactctctgtctccaagtctggcacctcagcctccctggccatcagtgggctccagtctgagcatgaggctgattattactgtgcagcatgggatgacagcctgaatggttttgtggtc

SEQ ID NO: 11 - protein, 105 aa, Artificial Sequence
  Synthetic Peptide
LVQSGAEVKKPGESLKISCKGSGYSFTSYWIGWVRQMPGKGLEWMGIIYPGDSDTRYSPSFQGQVTISADKSISTAYLQWSSLKASDTAMYYCARRGVRGSPFDY

SEQ ID NO: 12 - DNA, 315 bp, Artificial Sequence
  Synthetic Primer
ctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgaggcggggggttcggggatccccttttgactac

SEQ ID NO: 13 - DNA, 315 bp, Artificial Sequence
  Synthetic Primer
ctggtgcagtctggagcagaggtgaaaaagcccggggagtctctgaagatctcctgtaagggttctggatacaggttttccagctactggatcgcctgggtgcgccacgtgcccgggaaaggcctggagtggatgggtatcatttatcctggttactctgaaaccagatacaacccgtcgttccaaggccacgtcaccatctcttccgacaagtctaatggcaccgcctacctgcagtggagcagactgagggcctcggacaccgccatgtattactgtgcgaggcggggggatgggggatccccttttgacaac

SEQ ID NO: 14 - DNA, 315 bp, Artificial Sequence
  Synthetic Primer
ctggtgcagtctggagcagaggtgaaaaagcccggggagtctctgaagatctcctgtaagggttctggatacaggttttccagctactggatcgcctgggtgcgccacgtgcccgggaaaggcctggagtggatgggtatcatttatcctggttactctgaaaccagatacaacccgtcgttccaaggccacgtcaccatctcttccgacaagtctaatggcaccgcctacctgcagtggagcagactgagggcctcggacaccgccatgtattactgtgcggggcggggggatgggggatccccttttgacaac

SEQ ID NO: 15 - DNA, 315 bp, Artificial Sequence
  Synthetic Peptide
ctggtgcagtctggagcagaggtgaagaagcccggggagtctctgaagatctcctgtaagggttctggatacaggttttccagctactggatcgcctgggtgcgccacgtgcccgggaaaggcctggagtggatgggtatcatttatcctggttactctgaaaccagatacaacccgtcgtcccaaggccacgtcaccatctcttccgacaagtctaatggcaccgcctacctgcagtggagcagactgagggcctcggacaccgccatgtattactgtgcgaggcggggggatgggggatccccttttgacaac

SEQ ID NO: 16 - DNA, 315 bp, Artificial Sequence
  Synthetic Primer
ctggtgcagtctggagcagaggtgaaaaagcccggggagtctctgaagatctcctgtaagggttctggatacaggttttccagctacttgatcgcctgggtgcgccacgtgcccgggaaaggcctggagtggatgggtatcatttatcctggttactctgaaaccagatacaacccgtcgttccaaggccacgtcaccatctcttccgacaagtctaatggcaccgcctacctgcagtggagcagactgagggcctcggacaccgccatgtattactgtgcgaggcgggggggtgggggatccccttttgacaac

SEQ ID NO: 17 - DNA, 315 bp, Artificial Sequence
  Synthetic Primer
ctggtgcagtctggagcagaggtgaaaaagcccggggagtctctgaagatctcctgtaagggttctgggtacaggttttccagctactggatcgcctgggtgcgccacgtgcccgggaaaggcctggagtggatgggtatcatttatcctggttactctgaaaccagatacaacccgtcgttccaaggccacgtcaccatctcttccgacaagtctaatggcaccgcctacctgcagtggagcagactgagggcctcggacaccgccatgtattactgtgcgaggcggggggatgggggatccccttttgacaac

SEQ ID NO: 18 - DNA, 315 bp, Artificial Sequence
  Synthetic Primer
ctggtgcagtctggggctgaggtgaaaaagcccggggagtctctgaagatctcctgtaagggttctggatacaggttttccagctactggatcgcctgggtgcgccacgtgcccgggaaaggcctggagtggatgggtatcatttatcctggttactctgaaaccagatacaacccgtcgttccaaggccacgtcaccatctcttccgacaagtctaatggcaccgcctacctgcagtggagcagactgagggcctcggacaccgccatgtattactgtgcgaggcggggggatgggggatccccttttgacaac

SEQ ID NO: 19 - DNA, 315 bp, Artificial Sequence
  Synthetic Primer
ctggtgcagtctggagcagaggtgaaaaagcccggggagtctctgaagatctcctgtaagggttctggatacaggttttccagctactggatcgcctgggtgcgccacgtgcccgggaaaggcctggagtggatgggtatcatttatcttggttactctgaaaccagatacaacccgtcgttccaaggccacgtcaccatctcttccgacaagtctaatggcaccgcctacctgcagtggagcagactgagggcctcggacaccgccatgtattactgtgcgaggcggggggatgggggatccccttttgacaac

SEQ ID NO: 20 - DNA, 315 bp, Artificial Sequence
  Synthetic Primer
ctggtgcagtctggagcagaggtgaaaaagcccggggagtctctgaagatctcctgtaagggttctggatacaggttttccagctactggatcgcctgggtgcgccacgtgcccgggaaaggcctggagtggatgggtatcatttatcctggtcactctgaaaccagatacaacccgtcgttccaaggccacgtcaccatctcttccgacaagtctaatggcaccgcctacctgcagtggagcagactgagggcctcggacaccgccatgtattactgtgcgaggcggggggatgggggatccccttttgacaac

SEQ ID NO: 21 - DNA, 315 bp, Artificial Sequence
  Synthetic Primer
ctggtgcagtctggagcagaggtgaaaaagcccggggagtctctgaagatctcctgtaagggttctggatacaggttttccagctactggatcgcctgggtgcgccacgtgcccgggaaaggcctggagtggatgggtatcatttatcctggttactctgaaaccagatacaacccgtcgttccaaggccacgtcaccatctcttccgacaagtctaatggcaccgcctacctgcagtggagcaggctgagggcctcggacaccgccatgtattactgtgcgaggcggggggatgggggatccccttttgacaac

SEQ ID NO: 22 - protein, 97 aa, Artificial Sequence
  Synthetic Peptide
DIQLTQSPSFLSASVGDRVTITCRASQGISSYLAWYQQKPGKAPKLLIYAASTLQSGVPSRFSGSGSGTEFTLTISSLQPEDFATYYCQQLNSYPLT

SEQ ID NO: 23 - DNA, 291 bp, Artificial Sequence
  Synthetic Primer
gacatccagttgacccagtctccatccttcctgtctgcatctgtaggagacagagtcaccatcacttgccgggccagtcagggcattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacagcttaatagttaccccctcact

SEQ ID NO: 24 - DNA, 291 bp, Artificial Sequence
  Synthetic Primer
gacatccagatgacccagtctccatccttcctgtctgcatctgtgggagacagagtcaccatcacttgccgggccagtcagggcattgccggttatttagcctggtatcagcaaaaaccaggcaaagcccctaaactcctgatatatgctgcatccactttacaaagtggggtcccgtcaaggttcagcggcagtggatcagggacagacttcactctcacaatcaacggcctgcagcctgaagattttgcaacttatcactgtcaacagcttaataatttccccctcact

SEQ ID NO: 25 - DNA, 291 bp, Artificial Sequence
  Synthetic Primer
gacatccagatgacccagtctccatccttcctgtctgcatctgtgggagacagagtcaccatcacttgccgggccagtcagggcattgccggttatttagcctggtatcagcaaaaaccaggcaaagcccctaaactcctgatatatgctgcatccactttacaaagtggggtcccgtcaaggttcagcggcagtggatcagggacagacttcactctcacaatcaacggcctgcagcctgaagattttgcaacttatcactgtcaacagcttaataatttcccccccact

SEQ ID NO: 26 - DNA, 291 bp, Artificial Sequence
  Synthetic Primer
gacatccagatgacccagtctccatccttcctgtctgcatctgtgggagacagagtcaccatcgcttgccgggccagtcagggcattgccggttatttagcctggtatcagcaaaaaccaggcaaagcccctaaactcctgatatatgctgcatccactttacaaagtggggtcccgtcaaggttcagcggcagtggatcagggacagacttcactctcacaatcaacggcctgcagcctgaagattttgcaacttatcactgtcaacagcttaataatttccccctcact

SEQ ID NO: 27 - DNA, 291 bp, Artificial Sequence
  Synthetic Primer
gacatccggatgacccagtctccatccttcctgtctgcatctgtgggagacagagtcaccatcacttgccgggccagtcagggcattgccggttatttagcctggtatcagcaaaaaccaggcaaagcccctaaactcctgatatatgctgcatccactttacaaagtggggtcccgtcaaggttcagcggcagtggatcagggacagacttcactctcacaatcaacggcctgcagcctgaagattttgcaacttatcactgtcaacagcttaataatttccccctcact

SEQ ID NO: 28 - DNA, 291 bp, Artificial Sequence; identical to SEQ ID NO: 27
  Synthetic Primer

SEQ ID NO: 29 - DNA, 291 bp, Artificial Sequence
  Synthetic Primer
gacatccggatgacccagtctccatcctccctgtctacatctgtgggagacagagtcaccatcacttgccgggccagtcagggcattgccggttatttagcctggtatcagcaaaaaccaggcaaagcccctaaactcctgatatatgctgcatccactttacaaagtggggtcccgtcaaggttcagcggcagtggatcagggacagacttcactctcacaatcaacggcctgcagcctgaagattttgcaacttatcactgtcaacagcttaataatttccccctcact

SEQ ID NO: 30 - DNA, 291 bp, Artificial Sequence
  Synthetic Primer
gacatccagatgacccagtctccatcttccgtgtctgcatctgtgggagacagagtcaccatcacttgccgggccagtcagggcattgccggttatttagcctggtatcagcaaaaaccaggcaaagcccctaaactcctgatatatgctgcatccactttacaaagtggggtcccgtcaaggttcagcggcagtggatcagggacagacttcactctcacaatcaacggcctgcagcctgaagattttgcaacttatcactgtcaacagcttaataatttccccctcact

SEQ ID NO: 31 - DNA, 291 bp, Artificial Sequence
  Synthetic Primer
gccatccagatgacccagtctccatcttccgtgtctgcatctgtgggagacagagtcaccatcacttgccgggccagtcagggcattgccggttatttagcctggtatcagcaaaaaccaggcaaagcccctaaactcctgatatatgctgcatccactttacaaagtggggtcccgtcaaggttcagcggcagtggatcagggacagacttcactctcacaatcaacggcctgcagcctgaagattttgcaacttatcactgtcaacagcttaataatttccccctcact

SEQ ID NO: 32 - DNA, 291 bp, Artificial Sequence
  Synthetic Primer
gacatccagatgacccagtctccatcctccctgtctgcatctgtgggagacagagtcaccatcacttgccgggccagtcagggcattgccggttatttagcctggtatcagcaaaaaccaggcaaagcccctaaactcctgatatatgctgcatccactttacaaagtggggtcccgtcaaggttcagcggcagtggatcagggacagacttcactctcacaatcaacggcctgcagcctgaagattttgcaacttatcactgtcaacagcttaataatttccccctcact

SEQ ID NO: 33 - DNA, 291 bp, Artificial Sequence
  Synthetic Primer
gacatccagatgacccagtctccatcctccctgtctacatctgtgggagacagagtcaccatcacttgccaggcgagtcaggacattgccaactatttaaattggtatcagcaaaaaccaggcaaagcccctaaactcctgatatatgctgcatccactttacaaagtggggtcccgtcaaggttcagcggcagtggatcagggacagacttcactctcacaatcaacggcctgcagcctgaagattttgcaacttatcactgtcaacagcttaataatttccccctcact

SEQ ID NO: 34 - protein, 114 aa, Artificial Sequence
  Synthetic Peptide
VQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDQVVTGTQFYYYYGMDV

SEQ ID NO: 35 - DNA, 342 bp, Artificial Sequence
  Synthetic Primer
gtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagagatcaggtggtgactgggacccagttctactactactacggtatggacgtc

SEQ ID NO: 36 - DNA, 342 bp, Artificial Sequence
  Synthetic Primer
gtgcagctggtgcagtctggggctgaactgaagaagcctggggcctcagtgaaggtctcctgcagggcttctggagacaccttcaccgactactatatgcactgggtccgacagtcccctggacaagggcttgagtggatgggttgggtcaaccctaacagtggtggcacaaactacgcacagaagtttctgggcagggtcaccttgaccagggacacgtccatcagcacagtctacatggaactgagtaggcttagatctgacgacacgggcgtatattattgtgcgagagatcagggggagactgggacccagttctactactactatggtatggatgtc

SEQ ID NO: 37 - DNA, 342 bp, Artificial Sequence
  Synthetic Primer
gtgcagctggtacagtctggggctgaactgaagaagcctggggcctcagtgaaggtctcctgcagggcttctggagacaccttcaccgactactatatgcactgggtccgacagtcccctggacaagggcttgagtggatgggttgggtcaaccctaacagtggtggcacaaactacgcacagaagtttctgggcagggtcaccttgaccagggacacgtccatcagcacagtctacatggaactgagtaggcttagatctgacgacacgggcgtatattattgtgcgagagatcagggggagactgggacccagttctactactactatggtatggatgtc

SEQ ID NO: 38 - DNA, 342 bp, Artificial Sequence
  Synthetic Primer
gtgcagctggtacagtctggggctgaactgaagaagcctggggcctcagtgaaggtctcctgcagggcttctggagacgccttcaccgactactatatgcactgggtccgacagtcccctggacaagggcttgagtggatgggttgggtcaaccctaacagtggtggcacaaactacgcacagaagtttctgggcagggtcaccttgaccagggacacgtccatcagcacagtctacatggaactgagtaggcttagatctgacgacacgggcgtatattattgtgcgagagatcagggggagactgggacccagttctactactactatggtatggatgtc

SEQ ID NO: 39 - protein, 109 aa, Artificial Sequence
  Synthetic Peptide
VQLVQSGAEVKKPGSSVKVSCKASGGTFSSYAISWVRQAPGQGLEWMGGIIPIFGTANYAQKFQGRVTITADKSTSTAYMELSSLRSEDTAVYYCATEDDYSTTGHIDY

SEQ ID NO: 40 - DNA, 327 bp, Artificial Sequence
  Synthetic Primer
gtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacaaatccacgagcacagcctacatggagctgagcagcctgagatctgaggacacggccgtgtattactgtgcgacagaagatgactacagtacaacgggacacatagactac

SEQ ID NO: 41 - DNA, 327 bp, Artificial Sequence
  Synthetic Primer
gtgcagctggtgcagtctggggctgaggtgaagaagcctgggtcctcggtgaaggtctcctgcaaggcttctggaggcaccttcatcacctttggtctgaactggctgcgacaggcccctggacaagggcttgagtggatgggagtgatcatccccatcgttgagagagtgaactacgcagagaggttgcagggcagagtcacgataaccgcggacaagtccacgaacactgcctacctggagctgagcagcctgaaagctgaggacacggccgtgtattactgtgcgacagaagatgactacagtacaacgggacacatagacttc

SEQ ID NO: 42 - DNA, 327 bp, Artificial Sequence
  Synthetic Primer
gtgcagctggtgcagtctggggctgaggtgaagaagcctgggtcctcggtgaaggtctcctgcaaggcttctggaggcaccttcatcacctttggtctgaactggctgcgacaggcccctggacaagggcttgagtggatgggagtgatcatccccatcgttgagagagtgaactacgcagagaggttgcagggcagagtcacgataaccgcggacaagtccacgaacactgcctacctggagctgagcagcctgaaagctgaggacacggccgtgtattactgtgcgacagaagatgactacagtacgacgggacacatagacttc

SEQ ID NO: 43 - DNA, 327 bp, Artificial Sequence
  Synthetic Primer
gtgcagctggtacagtctggggctgaggtgaagaagcctgggtcctcggtgaaggtctcctgcaaggcttctggagacaccttcatcacctttggtctgaactggctgcgacaggcccctggacaagggcttgagtggatgggagtgatcatccccatctttgagagagtgaactacgcagagaggttgcagggcagagtcacgataaccgcggacaagtccacgaacactgcctacctggagctgagcagcctgaaagctgaggacacggccgtgtattactgtgcgacagaagatgactacagtacaacgggacacatagacttc

SEQ ID NO: 44 - protein, 97 aa, Artificial Sequence
  Synthetic Primer
DIQMTQSPSTLSASVGDRVTITCRASQSISSWLAWYQQKPGKAPKLLIYDASSLESGVPSRFSGSGSGTEFTLTISSLQPDDFATYYCQQYNSYSPT

SEQ ID NO: 45 - DNA, 291 bp, Artificial Sequence
  Synthetic Primer
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctatgatgcctccagtttggaaagtggggtcccatcaaggttcagcggcagtggatctgggacagaattcactctcaccatcagcagcctgcagcctgatgattttgcaacttattactgccaacagtataatagttattctcccact

SEQ ID NO: 46 - DNA, 291 bp, Artificial Sequence
  Synthetic Primer
gacatccagatgacccagtctccttccaccctgtctgcatctataggagacagagtcaacattacttgccgggccagtcagagtattggtaactggttggtctggtatcagcacaaaccagggaaagcccctaagttccttatctatgatgcctccactttgcacagtggggtcccatcaaggttcagcggcagtggatctgggacagaattcactctcaccatcagcagcctgcagcctgatgattttgcaacttattactgccaagagtataaaagtgattctcccact

SEQ ID NO: 47 - DNA, 291 bp, Artificial Sequence
  Synthetic Primer
gacatccagatgacccagtctccttccaccctgtctgcatctataggagacagagtcaacattacttgccgggccagtcagagtattggtaactggttggtctggtatcagcacaaaccagggaaagcccctaagttccttatctatgatgcctccactttgcgcagtggggtcccatcaaggttcagcggcagtggatctgggacagaattcactctcaccatcagcagcctgcagcctgatgattttgcaacttattactgccaagagtataaaagtgattctcccact

SEQ ID NO: 48 - DNA, 291 bp, Artificial Sequence
  Synthetic Primer
gacatccggatgacccagtctccttccaccctgtctgcatctataggagacagagtcaacattacttgccgggccagtcagagtattggtaactggttggtctggtatcagcacaaaccagggaaagcccctaagttccttatctatgatgcctccactttgcacagtggggtcccatcaaggttcagcggcagtggatctgggacagaattcactctcaccatcagcagcctgcagcctgatgattttgcaacttattactgccaagagtataaaagtgattctcccact

SEQ ID NO: 49 - protein, 97 aa, Artificial Sequence
  Synthetic Peptide
DIQMTQSPSSLSASVGDRVTITCQASQDISNYLNWYQQKPGKAPKLLIYDASNLETGVPSRFSGSGSGTDFTFTISSLQPEDIATYYCQQYDNLPWT

SEQ ID NO: 50 - DNA, 291 bp, Artificial Sequence
  Synthetic Peptide
gacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataatctcccttggacg

SEQ ID NO: 51 - DNA, 291 bp, Artificial Sequence
  Synthetic Primer
gacatccagatgacccagtctccatcctccctgtctacatctgtaggagacagagtcaccatcacttgccaggcgagtcaggacattagcaactatttaaattggtatcaacagaaaccagggaaagcccctaaactcctgatctccgatgcatccaatttggaaacaggggtcccatcaaggttcagtggaagtggatctgggacagattttactttcactatcagcagcctgcagcctgaagatattgcaacatattactgtcaacagtatgataatctcccttggacg

SEQ ID NO: 52 - DNA, 291 bp, Artificial Sequence
  Synthetic Primer
gacatccagatgacccagtctccatcctccctgtctacatctgtaggagacagagtcaccattacttgccaggcgagtcaggacattagcaactatttaaattggtatcaacagaaaccagggaaagcccctaaactcctgatctccgatgcatccaatttggaaacaggggtcccatcaaggttcagtggaagtggatctgggacagattttactttcactatcagcagcctgcagcctgaagatattgcaacatattactgtcaacagtatgataatctcccttggacg

SEQ ID NO: 53 - DNA, 291 bp, Artificial Sequence
  Synthetic Primer
gacatccagatgacccagtctccatcctccctgtctacatctgtaggagacagagtcaccatcacttgccaggcgagtcaggacattagcaactatttaaattggtatcaacagaaaccagggaaagcccctaaactcctgatctccgatgcatccaatttggaaacaggggtcccatcgaggttcagtggaagtggatctgggacagattttactttcactatcagcagcctgcagcctgaagatattgcaacatattactgtcaacagtatgataatctcccttggacg

SEQ ID NO: 54 - DNA, 291 bp, Artificial Sequence
  Synthetic Primer
gacatccggatgacccagtctccatcctccctgtctacatctgtaggagacagagtcaccatcacttgccaggcgagtcaggacattagcaactatttaaattggtatcaacagaaaccagggaaagcccctaaactcctgatctccgatgcatccaatttggaaacaggggtcccatcaaggttcagtggaagtggatctgggacagattttactttcactatcagcagcctgcagcctgaagatattgcaacatattactgtcaacagtatgataatctcccttggacg

SEQ ID NO: 55 - DNA, 291 bp, Artificial Sequence
  Synthetic Primer
gccatccggatgacccagtctccatcctccctgtctacatctgtaggagacagagtcaccatcacttgccaggcgagtcaggacattagcaactatttaaattggtatcaacagaaaccagggaaagcccctaaactcctgatctccgatgcatccaatttggacacaggggtcccatcaaggttcagtggaagtggatctgggacagattttactttcactatcagcagcctgcagcctgaagatattgcaacatattactgtcaacagtatgataatctcccttggacg